SEQUENCE LISTING (37 sequences)

SEQ ID NO: 1 - DNA, 21 bp, Artificial Sequence
  Description of Combined DNA/RNA Molecule Synthetic oligonucleotide
gcaccgaaguauuguccaatt

SEQ ID NO: 2 - DNA, 21 bp, Artificial Sequence
  Description of Combined DNA/RNA Molecule Synthetic oligonucleotide
uuggacaauacuucggugctt

SEQ ID NO: 3 - protein, 14 aa, Artificial Sequence
  Description of Artificial Sequence Synthetic peptide
DILSEASTMQLLQQ

SEQ ID NO: 4 - protein, 14 aa, Artificial Sequence
  Description of Artificial Sequence Synthetic peptide
DIASEASTMQAAQQ

SEQ ID NO: 5 - DNA, 20 bp, Artificial Sequence
  Description of Artificial Sequence Synthetic primer
agccttcattctccaggacc

SEQ ID NO: 6 - DNA, 20 bp, Artificial Sequence
  Description of Artificial Sequence Synthetic primer
tcagacctccgtaagagtgc

SEQ ID NO: 7 - DNA, 20 bp, Artificial Sequence
  Description of Artificial Sequence Synthetic primer
agggatcagggagtctcaca

SEQ ID NO: 8 - DNA, 20 bp, Artificial Sequence
  Description of Artificial Sequence Synthetic primer
cgtcttgtcgttcacgatcg

SEQ ID NO: 9 - DNA, 20 bp, Artificial Sequence
  Description of Artificial Sequence Synthetic primer
tctctgggaggagaaagcag

SEQ ID NO: 10 - DNA, 20 bp, Artificial Sequence
  Description of Artificial Sequence Synthetic primer
cctatgtcctgactcccgtt

SEQ ID NO: 11 - DNA, 20 bp, Artificial Sequence
  Description of Artificial Sequence Synthetic primer
cgctatcagtcaccccattt

SEQ ID NO: 12 - DNA, 20 bp, Artificial Sequence
  Description of Artificial Sequence Synthetic primer
cgttagagtttcggaaaagc

SEQ ID NO: 13 - DNA, 20 bp, Artificial Sequence
  Description of Artificial Sequence Synthetic primer
tcgtggaacatgacaaggaa

SEQ ID NO: 14 - DNA, 20 bp, Artificial Sequence
  Description of Artificial Sequence Synthetic primer
ctgatccggaacgtctcatt

SEQ ID NO: 15 - DNA, 20 bp, Artificial Sequence
  Description of Artificial Sequence Synthetic primer
tggagatgctgtaccagtgc

SEQ ID NO: 16 - DNA, 20 bp, Artificial Sequence
  Description of Artificial Sequence Synthetic primer
agaggatgttttccggcttt

SEQ ID NO: 17 - DNA, 24 bp, Artificial Sequence
  Description of Artificial Sequence Synthetic primer
tttggagcagagaggaggcaatgg

SEQ ID NO: 18 - DNA, 25 bp, Artificial Sequence
  Description of Artificial Sequence Synthetic primer
tggtattaggatagaagcaccaggg

SEQ ID NO: 19 - DNA, 20 bp, Artificial Sequence
  Description of Artificial Sequence Synthetic primer
atcatgtttgagaccttcaa

SEQ ID NO: 20 - DNA, 20 bp, Artificial Sequence
  Description of Artificial Sequence Synthetic primer
catctcttgctcgaagtcca

SEQ ID NO: 21 - protein, 10 aa, Artificial Sequence
  Description of Artificial Sequence Synthetic peptide
XXXAXXXXLL

SEQ ID NO: 22 - protein, 10 aa, Artificial Sequence
  Description of Artificial Sequence Synthetic peptide
XXXXXXXXLL

SEQ ID NO: 23 - protein, 10 aa, Artificial Sequence
  Description of Artificial Sequence Synthetic peptide
HXXXXXXXLL

SEQ ID NO: 24 - protein, 11 aa, Artificial Sequence
  Description of Artificial Sequence Synthetic peptide
DHXXXXXXXLL

SEQ ID NO: 25 - protein, 10 aa, Artificial Sequence
  Description of Artificial Sequence Synthetic peptide
LXXXXXXXLL

SEQ ID NO: 26 - protein, 10 aa, Artificial Sequence
  Description of Artificial Sequence Synthetic peptide
AXXXXXXXAA

SEQ ID NO: 27 - protein, 5 aa, Artificial Sequence
  Description of Artificial Sequence Synthetic peptide
LXXLL

SEQ ID NO: 28 - protein, 10 aa, Homo sapiens
LSEASTMQLL

SEQ ID NO: 29 - protein, 10 aa, Mus sp.
LNEAGTMQLL

SEQ ID NO: 30 - protein, 10 aa, Rattus sp.
LSEAGTMQLL

SEQ ID NO: 31 - protein, 10 aa, Homo sapiens
HTKASGMALL

SEQ ID NO: 32 - protein, 10 aa, Mus sp.
HTKASGMALL

SEQ ID NO: 33 - protein, 10 aa, Rattus sp.
HTKASGMALL

SEQ ID NO: 34 - protein, 10 aa, Homo sapiens
LLAARTRQLL

SEQ ID NO: 35 - protein, 10 aa, Homo sapiens
LHSASGRGLL

SEQ ID NO: 36 - protein, 20 aa, Homo sapiens
YQAQDVGTGTLMAVKQVTYV

SEQ ID NO: 37 - protein, 30 aa, Homo sapiens
TNSGASAASRNMSGQVSMBPAFIHHHPPKS

(19) United States
(12) Patent Application Publication
(10) Pub. No.: US 2008/0269106 A1
(43) Pub. Date: Oct. 30, 2008

(54) Methods and Compositions for Diagnosis and Treatment of Disorders Involving Macrophages

(76) Inventors: Michael Rosenfeld, San Diego, CA (US); David W. Rose, San Diego, CA (US); Ping Zhu, San Diego, CA (US)

Correspondence Address: LATIMER, MAYBERRY & MATTHEWS IP LAW, LLP, 13873 PARK CENTER ROAD, SUITE 106, HERNDON, VA, 20171, US

(21) Appl. No.: 12/066,428

(22) PCT Filed: Sep. 12, 2006
(86) PCT No.: PCT/US06/35350

§ 371 Date: Mar. 11, 2008

Related U.S. Application Data
(60) Provisional application No. 60/715,575, filed on Sep. 12, 2005.

(51) Int. Cl. A61K 38/00; C12Q 1/00; C12Q 1/68; A61P 43/00; C12Q 1/04

(52) U.S. Cl. 514/2; 435/4; 435/34; 435/6

(57) ABSTRACT

The present invention discloses methods of identifying and treating diseases or disorders, particularly those relating to hormone receptors and their activities. The invention also discloses methods of screening for drugs that can be used to treat diseases or disorders. The invention is based, at least in part, on the discovery of the role of macrophages in development of certain cancers, such as prostate cancer and breast cancer.

### CROSS-REFERENCE TO RELATED APPLICATIONS

This application claims the benefit of, and relies on the filing date of, U.S. provisional patent application No. 60/715,575, filed 12 Sep. 2005, the entire disclosure of which is hereby incorporated herein by reference.

### STATEMENT OF GOVERNMENT INTEREST

This invention was made partially with U.S. Government support from the United States Department of Health and Human Services, National Institutes of Health, National Institute of Diabetes & Digestive & Kidney Diseases, under Contract No. 5R37DK39949. The U.S. Government has certain rights in the invention.

### BACKGROUND OF THE INVENTION

1. Field of the Invention

The present invention relates to the field of the biology of diseases and disorders. More specifically, the present invention pertains to diseases or disorders caused by a macrophage induced signaling pathway. The invention pertains to causes of cancers and other diseases or disorders, methods of detecting, monitoring, or prognosing cancers or other diseases or disorders, and methods of treating cancers and other diseases and disorders, such as diabetes and atherosclerosis. It also relates to screening for drugs that can be used to treat cancers and other diseases and disorders.

2. Description of Related Art

The pattern of transcriptional response to the multiple signaling factors impacting each cell reflects, in part, an ability to generate an integrated transcriptional response program of gene activation and repression. The actions of nuclear receptors have provided an ideal model in which to investigate this question, as androgen and estrogen receptors can bind both to agonists, such as dihydrotestosterone (DHT) and 17-β-estradiol (E₂), or to selective androgen and estrogen receptor modulators (SARMs, SERMs), which act as antagonists or as weak agonists in a context-dependent fashion.

Androgens, acting via androgen receptors, are essential for normal growth and function of the prostate gland (Chunha et al., 1987). In the prostate gland, and in prostate cancer, regulation of cell growth is subserved by the liganded androgen receptor. Androgens are required in all animal models of prostate carcinogenesis, and androgen ablation is a standard for treatment for metastatic disease (Dedes and Tindall, 2004; Feldman and Feldman, 2001), but there is an invariant progression from androgen-dependent to androgen-independent growth, even though high levels of androgen receptor generally continue to be present (Chen et al., 2004; Dedes and Tindall, 2004; Feldman and Feldman, 2001). Resistance to anti-androgen treatment has been postulated to reflect diverse mechanisms, such as altered expression of other oncogene suppressors or increased expression of anti-apoptotic genes, alterations of levels of nuclear receptor cofactors, silencing of androgen receptor gene mutations in the AR sequence, and increased levels of androgen receptor (Chen et al., 2004; Dedes and Tindall, 2004; Feldman and Feldman, 2001). But, each of these could only account for a subset of resistance events. However, whether and how external signaling from the microenvironments of tumors might affect hormone resistance has been largely unknown.

The androgen receptor functions as a ligand-inducible transcription factor that regulates the expression of target genes having an androgen response element (Feldman and Feldman, 2001; Heinlein and Chang, 2002; Balk, 2002). Analogous to other members of the nuclear receptor superfamily, androgen receptor actions require AF-2 transcription activation function and an extended N-terminal domain with a strong AF-1 function (Berry et al., 1990; Onate et al., 1998; Bevan et al., 1999; He et al., 2002). In addition, there are interactions between the N- and C-terminal domains in steroid hormone activation and recruiting of many cofactors important for the transcriptional regulation (Kraus et al., 1995; Langley et al., 1995; Kemppainen et al., 1999; Tetel et al., 1999; Le Douarin et al., 1995; Heery et al., 1997; Torchia et al., 1997; Hong et al., 1997; Ding et al., 1998; Voegel et al., 1998; Darimont et al., 1998; McInerney et al., 1998). Structural studies of estrogen and androgen receptors and coactivator recognition have revealed, in concert with other receptors, that agonists and antagonists induce different conformations of helix 12, with antagonists blocking AF-2 function by preventing formation of an effective “charge clamp” for the LXXLL interaction motif (Renaud et al., 1995; Wagner et al., 1995; Brzozowski et al., 1997; Darimont et al., 1998; Nolte et al., 1998; Shiau et al., 1998; Moras and Gronemeyer, 1998; Wagner et al., 1995; Brzozowski et al., 1997; Darimont et al., 1998; Nolte et al., 1998; Shiau et al., 1998).

Investigation of active repression of gene expression by unliganded nuclear receptors has led to the identification of the nuclear receptor corepressor (N-CoR) (Horlein et al., 1995) and a closely related silencing mediator for retinoic acid and thyroid hormone receptor (SMRT) (Chen and Evans, 1995; Sande and Privalsky, 1996), which contain multiple repressor domains that could transfer their active repression function, recruiting histone deacetylases (HDACs). Based on genetic analyses, N-CoR proves to be required for repression by T₃R, RAR, and other nuclear receptors, and for the inhibitory function of estrogen receptor antagonists (SERMs) (Jackson et al., 1997, Smith et al., 1997; Lavinsky et al., 1998; reviewed in McKenna et al., 1999; Glass and Rosenfeld et al., 2000). N-CoR has been found as a component of many complexes, including TBL1/TBLR1/HDAC3/GPS2 (Guenther et al., 2000; Li et al., 2000; Wen et al., 2000; Zhang et al., 2002; Yoon et al., 2003), TAB2/HDAC3 (Baek et al., 2002), Sin3/HDAC1, 2 complexes (Heinzel et al., 1997; Nagy et al., 1997; Alland et al., 1997), complexes containing SWI/SNF-related proteins (Underhill et al., 2000), and other multiple complexes (Jepsen and Rosenfeld, 2002). Sequences referred to as the CoRNR box (Hu and Lazar, 1999) or alternatively as LXXX IXXX I/L motifs (Nagy et al., 1999; Perissi et al., 1999), appear to bind in the hydrophobic pocket that is occupied by the coactivator LXXLL helical motifs upon binding of ligand, but conformational effects (Renaud et al., 1995; Wagner et al., 1995; Brzozowski et al., 1997; Darimont et al., 1998; Nolte et al., 1998; Shiau et al., 1998; Moras and Gronemeyer, 1998; Wu et al., 2005) of ligand binding inhibits the binding of corepressors, implicating the initiating molecular mechanism for ligand-dependent displacement of the corepressor complex. Thus, the N-CoR complex/complexes are prototypical of the large number of multicomponent complexes that activate or repress target genes.

Recently, it has been recognized that there is an increased macrophage infiltration in the adipose tissue of individuals who are obese, and that macrophages might be a major source of inflammatory cytokines in adipose tissue (Weisberg et al., 2003; Xu et al., 2003; Arkan et al., 2005; Cai et al., 2005). This interaction may contribute to insulin resistance by the actions of pro-inflammatory cytokines (Arkan et al., 2005; Cai et al., 2005). The potential role of macrophage-related pro-inflammatory signals is of great interest in developmental biology and in disease, particularly cancers. Based on analysis of IKKβ gene deleted mice, interruption of the IKK/NF-κB pathway attenuates inflammation-associated tumors (Greten et al., 2004). Among their phenotypic actions, pro-inflammatory signals can de-repress genes regulated by the p50 homodimer of NF-κB DNA binding factors by dismissal of the N-CoR complex (Baek et al., 2002). It is thus of particular interest to determine whether a macrophage/prostate cancer cell interaction occurs, if it is a common event in prostate cancer, and whether it serves to elicit inflammatory signals capable of impacting the therapeutic effectiveness of SARMs. These compounds typically initially function as antagonists in vivo, but almost invariantly become ineffective over a period of time (Grese et al., 1997; Shang et al., 2002b, Smith and O'Malley, 2004).

U.S. patent application publication number 2003/0040050 discloses that TAB2 is a molecule that mediates signal transduction of IL-1. In this publication, inhibitors of TAB2 have been suggested to be useful as anti-inflammatory drugs, working through their action on early signaling events relating to the IL-1 receptor. There is no suggestion in the patent application publication of a link between macrophages and cancer, or that macrophages may elicit biologically significant levels of IL-1 outside of the immune system.

Although much progress has been made in deciphering the molecular bases of cancers and other diseases and disorders, there still exists a need for further insights into causation and treatment of these ailments. There is likewise a need for a better understanding of the development of diseases and disorders and their interactions with drugs used to treat them. For example, there is a need in the art for a method to block compounds used against prostate cancer from becoming ineffective.

### SUMMARY OF THE INVENTION

The present invention addresses needs in the art by providing methods, compounds, and compositions, for treating diseases and disorders, such as, but not limited to, cancers, diabetes, and atherosclerosis. The invention is based, at least in part, in the surprising recognition that macrophages can promote development of diseases and disorders through a signaling pathway that was previously unrecognized as being affected by macrophages, and which involves TAB2. In this pathway, macrophage interactions with cells activate pro-inflammatory signals, causing a cascade leading to a disease or disorder. In the case of cancer cells, this activation causes genetic changes that allow the cancer cells to become resistant to anti-cancer drugs. The present invention shows that these genetic changes are mediated by TAB2, a molecule that is recruited to the N-CoR complex, leading to its relocation outside of the cell nucleus, resulting in derepression of genes regulated by hormone receptors. Therefore, this invention relates to hormone-related disorders such as those affecting sex or sex-specific cells.

The present invention also mechanistically links interactions between macrophages and normal reproductive biology for the first time. A key aspect of reproductive regulation, blastocyst implantation, involves a local induction of BMP7 in response to inflammatory signals. The invention shows the involvement of TAB2 in mediating inflammatory signal-dependent derepression of BMP7.

The present invention identifies macrophage-induced transcription of genes. The induction results in abnormal transcription of specific genes involved in various diseases and disorders. Accordingly, in one aspect, the invention provides a method of detecting the presence of a disease or disorder by detecting the presence of macrophages. In general, the method comprises detecting the presence of abnormal levels of macrophages in biological tissue. Typically, but not necessarily, the method comprises obtaining a sample of tissue to be assayed. In addition, the method often comprises assaying for TAB2, alone or in complexes with other proteins, such as receptors or transcription factors. Often, the sample comprises biological tissue from a mammal, such as a human. Specific examples of diseases and disorders include, but are not limited to, cancer, diabetes, and atherosclerosis. Other examples include, but are not limited to, hormone-related disorders, such as those affecting sex-steroid receptors.

In another aspect, the invention provides a method of detecting the presence of a disease or disorder by detecting the presence of substances involved in a macrophage-induced signaling pathway. These substances include, but are not necessarily limited to, cytokines, VCAM1, MEKK1, TAB2, the N-CoR complex, and the L/HX₇LL motif. In general, the method comprises obtaining biological material of interest, and detecting one or more substance of interest, wherein detection of the substance indicates its presence in the biological material. Because the present invention provides a recognition of the cause of numerous diseases and disorders, based on a variety of signaling pathways that might have non-overlapping members, a wide variety of substances may be detected and be indicative of a disease or disorder. The pathways implicated by this invention are known, as are molecules that play a role in those pathways. Thus, one of skill in the art may easily select a substance for detection and use commonly available reagents (e.g., commercially available antibodies, common immunoassays or assays for protein-protein interactions, and the like) to achieve the detection, without the need for an exhaustive recitation herein of the techniques and reagents to use.

Defining the precise molecular strategies that coordinate patterns of transcriptional responses to specific signals is central for understanding normal development, homeostasis, and the pathogenesis of specific hormone-dependent cancers. The present invention discloses specific prostate cancer cell/macrophage interactions that mediate a switch in function of selective androgen receptor antagonists/modulators (SARMs) from repression to activation in vivo. This conversion is based on the presence of a conserved receptor N-terminal L/HX₇LL motif, selectively present in sex steroid receptors. This motif is responsible, at least in part, for recruitment of TAB2 as a component of an N-CoR co-repressor complex. TAB2 acts as a sensor for inflammatory signals by acting as a molecular beacon for recruitment of MEKK1, which in turn mediates dismissal of the N-CoR complex, permitting derepression of the androgen and estrogen receptors. Surprisingly, this conserved sensor strategy might have arisen to mediate reversal of sex steroid-dependent repression of specific target genes, such as BMP7, in response to inflammatory signals, physiologically linking inflammatory and nuclear receptor ligand responses to essential replicative functions.

In this invention, it is established that specific interactions between cells and macrophages occur that lead to a disease or disorder. For example, it is established that specific interactions between prostate cancer cells and macrophages occur, and the interaction promotes cancerous growth of the prostate cells. In general, the interactions result in conversion of SARMs from antagonists of gene expression to agonists of gene expression by activating a functional program of specific pro-inflammatory signals that cause dismissal of the N-CoR holocorepressor complex from androgen and estrogen receptors. The conversion is based, at least in part, on the presence of TAB2, which is recruited to the complex based on the actions of a specific, evolutionarily-conserved, N-terminal L/HX₇LL motif found in receptors, such as sex steroid receptors. This motif serves as a discriminatory molecular beacon for specific pro-inflammatory cytokine signals, modulating a coordinated program of gene derepression. A signal from IL-1β, for example, results in derepression of gene targets for sex steroids and NF-κB, but not for the other classes of nuclear receptors, thus permitting an integrated pattern of transcriptional response. One evolutionary basis for this appears to be the importance of inflammatory signal-dependent derepression of sex steroid-repressed genes in reproductive biology.

In view of the information presented herein, in another aspect, the present invention provides methods of treating diseases or disorders. In general, the methods comprise administering to a subject in need at least one substance that affects the interaction between at least one macrophage-induced substance and expression of at least one gene involved in a disease or disorder. Typically, the method involves administering a substance or combination of substances that affect the interaction of two or more proteins that are involved in one or more signaling pathways between one or more cytokines and expression of a gene. This method can involve administering an effective amount of an inhibitor of MEKK1 expression, VCAM1 expression, or TAB2 expression. This method can also involve administering an effective amount of a substance that affects the activity of a gene product involved in this pathway, such as the activity of MEKK1, VCAM1, TAB2, and/or one or more gene products involved in the N-CoR complex. This method can also or alternatively include administering an effective amount of a substance that interferes with the interaction or binding between two or more molecules. The molecules can comprise macrophages and VCAM1, TAB2 and an antagonist/NCoR complex, the binding of a cytokine to another molecule, or the binding of a L/HX₇LL motif to another molecule. The method can also comprise administering to a subject an effective amount of a substance that interferes with the interaction between macrophages and cells of the disease or disorder. In some embodiments, the method is practiced in vitro as a research method.

In another aspect, the invention provides methods of screening for substances that are effective at treating at least one disease or disorder. In general, the methods comprise exposing at least one gene or gene expression product to a substance, and determining if the substance affects the expression of the gene or the activity of the gene expression product. The method of screening can comprise using an expression or activity inhibition assay such as a MEKK1 assay, a VCAM1 assay, a TAB2 assay, a N-CoR complex assay, or a L/HX₇LL motif assay. The method can also comprise exposing cells to a substance and determining if the substance affects the ability of macrophages to interact with the cells. In embodiments, the method is a method of identifying one or more substances that are effective at treating a cancer, such as one involving improper activation of gene expression as a result of the activity of a nuclear receptor and/or transcription factor, or one involving gene activation by a hormone receptor.

The present invention also provides a method of screening for substances that inhibit, reduce, or abolish the effects of macrophage-derived substances on expression of genes controlled by hormone receptors. In these methods, N-CoR−/− cells are used. The cells may be used in culture (i.e., in vitro) or may be used in an in vivo model (i.e., a knock-out animal, such as a knock-out mouse, rat, rabbit, etc.). In general, the method comprises contacting one or more N-CoR−/− cells and one or more substance, and determining if a change in expression of one or more genes occurs as a result of the contacting. In preferred embodiments, a change in a gene controlled by a hormone receptor is determined. Any of the various known techniques for determining the amount of gene expression in vivo or in vitro may be used. As with other methods that involve identifying, detecting, determining, etc. levels of one or more substance before and after a treatment, it is preferred that a control reaction be performed to provide a baseline from which a change can be determined. Those of skill in the art are well aware of the types of control reactions that may be performed for each of the methods disclosed herein, and thus each control reaction (whether positive or negative) need not be detailed herein. According to this aspect of the invention, the invention provides a method of detecting and/or identifying one or more substances that, when administered to a subject, treat at least one disease and/or disorder by affecting interaction of at least one macrophage with one or more disease cells or cells that can be converted to disease cells by such interaction.

In yet another aspect, the invention provides methods to increase or decrease fertility. The methods can rely on using anti-antigens with TAB2. More specifically, the invention discloses that the mechanisms involved in hormone receptor-mediated gene expression are conserved, at least to some extent, in reproductive biology. For example, the mechanism is active at the implantation stage of a blastocyst in the uterine lining. By regulating the effects of cytokines on the blastocyst and maternal cells, implantation can be enhanced or reduced, resulting in an increase or decrease in rate of successful impregnation. In general, the method of this aspect of the invention comprises contacting a blastocyst or the area surrounding the blastocyst with at least one compound that inhibits or promotes infiltration of macrophages into the area of the blastocyst, such as the uterine lining surrounding the blastocyst (e.g., within one to one hundred cells, inclusive, of the blastocyst implantation site). In embodiments, the method comprises contacting a blastocyst or the area surrounding the blastocyst with at least one compound that inhibits or promotes contact of at least one macrophage and at least one cell of the blastocyst.

In a further aspect, the invention provides methods to detect or decrease insulin resistance. Insulin resistance might be caused in part by the actions of inflammatory cytokines, such as IL-1β (Cai et al., 2005). These inflammatory cytokines are thought to be present as a result of an increased monocyte/macrophage infiltration in adipose tissue of obese individuals (Weisberg et al., 2003; Xu et al., 2003; Arkan et al., 2005; Cai et al., 2005). The present invention discloses that binding of TAB2 to complexes comprising hormone receptors is important in macrophage-induced resistance to anti-cancer substances. The macrophage-induced resistance relies, at least in part, on the presence of TAB2 in the complex. Given the similarity between the insulin resistance and the resistance to anti-cancer substances, it therefore follows that inhibition of TAB2 or other inhibitions discussed provides a method of detecting and/or decreasing insulin resistance.

Another aspect of the invention relates to atherosclerosis. Chronic inflammation characterizes atherosclerosis. Lesions as a result of the disease contain immune cells, particularly macrophages and T cells (reviewed in Li and Glass, 2002). Cytokines augment expression of the genes encoding various leukocyte adhesion molecules, including VCAM-1. The present invention discloses, for the first time, that the TAB2 molecule may be used as a means of detecting atherosclerosis. Accordingly, in this aspect, the invention provides a method of detecting atherosclerosis, where the method comprises obtaining biological material from suspected atherosclerotic tissue, and determining if TAB2 is present in the tissue, the presence of TAB2, alone or in complexes with other proteins, being indicative of an atherosclerotic state in the tissue. Further, inhibition of TAB2 or other inhibitions described for this invention may inhibit atherosclerosis. Accordingly, the invention provides a method of inhibiting progression, and preferably reversal of, atherosclerosis. The method comprises administering to a subject in need thereof an effective amount of an inhibitor of TAB2, of TAB2 phosphorylation, or another inhibitor of macrophage-induced transcription or macrophage adherence to cells.

In a further aspect, compositions comprising substances that cause a change in macrophage induced transcription of genes are provided. In general, a composition of the invention comprises a sufficient amount of at least one substance to cause a detectable change in transcription of at least one gene. Thus, when used in methods of treating, the compositions comprise at least one substance in an amount effective to cause a detectable change in macrophage induced transcription of at least one gene in a cell of a subject. In embodiments, the substances are provided in an amount sufficient to provide one or more doses to a subject. Certain other aspects of the invention provide for use in the preparation of compositions for medical use, such as pharmaceutical or therapeutic compositions. Compositions may comprise substances of the invention along with one or more other substances, which are typically substances that are biologically tolerable in that they may be exposed to living cells at their useful concentrations without killing the cells. In embodiments, the other substances are pharmaceutically acceptable substances.

Another aspect of the invention provides a container comprising one or more substances that cause a change, such as a decrease, in the macrophage induced transcription of genes. In general, where designed for in vivo treatment of a subject, a container according to the invention contains a sufficient amount of substance to provide at least one dose to the subject. In some embodiments, kits comprising one or more containers are provided.

### BRIEF DESCRIPTION OF THE DRAWINGS

The accompanying drawings, which constitute a part of this specification, illustrate several embodiments of the invention and, together with the written description, serve to explain various principles of the invention.

FIGS. 1A-1E are photographs and graphs showing the VCAM1-dependent interaction of macrophages and prostate cancer cells.

FIGS. 2A-2G are graphs and gels showing that N-CoR is a corepressor in hormone receptor-containing complexes.

FIGS. 3A-3G are arrays, graphs, and gels showing the effects of IL-1β on antagonists of hormone receptor gene transcription function.

FIGS. 4A-4H are graphs, whole cell immunofluorescence results, and gels showing the effects of IL-1β on antagonists of hormone receptor gene transcription function.

FIGS. 5A-5J are gels, graphs, and sequence comparisons showing the effects of a conserved amino acid motif, TAB2, and IL-1β on gene expression.

FIG. 6A-6E are gels, graphs, and a cartoon depicting the physiological roles of inflammatory signal dependent derepression in sex steroid receptor regulation.

FIG. 7A-7D are graphs depicting the combinatorial functions of HDACs in SARM-dependent repression.

FIG. 8A-8H are gels and graphs showing evaluations of the roles of certain co-activators.

FIG. 9A-9C are graphs showing the role of the LXXLL motif of SRC-1 in agonist-mediated activation and IL-1β-induced derepression of the androgen receptor.

FIG. 10A-10B are gels showing the roles of the N-terminal L/HX₇LL motif of steroid hormone receptors and MEKK1 in the interactions of TAB2/nuclear receptors and TAB2/N-CoR.

### DETAILED DESCRIPTION OF VARIOUS EMBODIMENTS OF THE INVENTION

Reference will now be made in detail to various exemplary embodiments of the invention, examples of which are illustrated in the accompanying drawings. The following detailed description is provided to give details on certain embodiments of the invention, and should not be understood as a limitation on the full scope of the invention.

Broadly speaking, the present invention provides methods, compounds, and compositions for treating diseases and disorders caused by abnormal transcription of genes. Abnormal transcription includes both an increase and a decrease in transcription as compared to the transcription of normal or non-diseased cells or tissue. The present invention has use in treating diseases and disorders caused by abnormal transcription of genes. It thus has both in vivo and in vitro uses.

In one aspect, the invention provides a method of detecting the presence of a disease or disorder by detecting the presence of macrophages in a sample comprising biological material. The method comprises: obtaining a sample, such as one comprising biological material, and detecting the presence of macrophages or abnormal levels of macrophages in the sample, the presence of macrophages or abnormal levels of macrophages in the sample being indicative of a disease or disorder. Preferably, a control reaction is run, or the results are compared to a sample of the same tissue that is known to be healthy tissue, in order to be better able to determine if an unusual or abnormal number of macrophages, or a significant change in macrophage numbers, is present in the test sample. In embodiments, the sample is a sample of cancerous tissue, diabetic tissue, or atherosclerotic tissue. In embodiments, the sample comprises one or more prostate cells or comprises prostate tissue, and the method detects the presence of, or the pre-disposition to development of, prostate cancer. In embodiments, the sample comprises one or more breast cell or comprises breast tissue, and the method detects the presence of, or the pre-disposition to development of, breast cancer. According to this aspect of the invention, the invention provides for the use of macrophages as an indicator of a disease or disorder state. It thus provides for the use of macrophages in identifying and treating a disease or disorder, such as a cancer involving one or more hormone receptors.

In another aspect of the invention, one or more molecules involved in a macrophage-induced signaling pathway can be used to detect the presence of a disease or disorder. In general, the method comprises: obtaining a sample, such as one comprising biological material, and detecting the presence of one or more substances (e.g., molecules) that are involved in a macrophage-induced signaling pathway, the presence of one or more such molecules or abnormal levels of such molecules in the sample being indicative of a disease or disorder. The molecules to be detected include, without limitation, one or more cytokines, VCAM1, MEKK1, TAB2 (phosphorylated or unphosphorylated), the N-CoR complex or higher-level complexes comprising N-CoR, and peptides, polypeptides, and proteins comprising the L/HX₇LL amino acid motif. Typically, the method comprises obtaining a biological sample and detecting the presence of abnormal levels of at least one of the molecules in the signaling pathway. In embodiments, the method can detect more than one kind of molecule. This aspect has use in treating a disease or disorder by detecting the presence of one or more molecules involved in a disease or disorder involving or resulting from a macrophage-induced signaling pathway. It thus provides for the use of these molecules in identifying and treating a disease or disorder, such as a cancer involving one or more hormone receptors.

In addition to detecting cancers, the methods of detecting a disease or disorder can detect a disease or disorder that is related to inflammation. Specifically, the methods involve a macrophage-induced signaling pathway, a pathway which is not limited to any particular disease or disorder, but which is involved in many different recognized diseases and disorders. Indeed, the realization that interaction between macrophages and other cells causes the other cells to change from healthy, normal cells to disease or disorder cells, and the discovery of the underlying mechanism, has broad implications in the field of treatment of diseases and disorders, and in drug discovery.

In view of the breadth of cells, diseases, and disorders encompassed by the present invention, it should be recognized that all cells for which a macrophage-induced signaling pathway may be present are contemplated by the present invention. Likewise, all diseases and disorders for which a macrophage-induced signaling pathway may be found to be involved in the disease or disorder are contemplated by the present invention. For example, the disease or disorder may be, but is not limited to, any type of cancer, diabetes, and atherosclerosis. In embodiments, the disease or disorder is a hormone-related disorder, such as one affecting or involving sex-steroid receptors. The disease or disorder can be hormone-related and therefore is relevant to cells involved in sexual differentiation or related to one sex. These cells include spermatozoa, ovum, breast, prostate, ovarian, and endometrium cells. Accordingly, in one aspect, the invention provides a method of detecting the presence of, the development of, or the possible development of, cancer by detecting the presence of macrophages in tissues. In general, the method comprises obtaining a sample of cancer tissue, tissue suspected of being cancer tissue, or tissue suspected of being susceptible to development of a cancerous state, and detecting the presence of macrophages in the tissue, the presence of macrophages or an abnormal level of macrophages indicating that the tissue is cancer tissue, is developing into cancer tissue, or is susceptible to development of cancer tissue. In embodiments, the tissue is prostate tissue and the cancer is prostate cancer. In other embodiments, the tissue is breast tissue and the cancer is breast cancer.

The sample to be used in the methods is not particularly limited in constitution, origination, or size. For example, it may be a biological sample obtained from an individual (also referred to herein interchangeably as a subject or patient, terms that include both human and non-human animals), such as blood and other liquid samples (e.g., blood products, macerated or liquified solid tissues, and the like) or solid tissue samples, such as a biopsy specimen or other tissue. The biological sample can thus encompass cells in culture, cell supernatants, cell lysates, serum, and plasma. One of skill in the art is fully capable of selecting the type and constitution of sample to be used, and adjusting the sample contents for particular assay conditions.

According to the invention, the term abnormal levels means a detectable and statistically significant increase or decrease in expression, transcription, activity, a measurable phenotypic characteristic or morphology, or any other characteristic that can be used to detect the presence of macrophages or other cells/molecules of interest. In embodiments, the increase or decrease is a substantial increase or decrease. Substantial increases or decreases means approximately a change of at least 20% from the normal or non-diseased levels, more preferably a change of at least about 40%, and even more preferably a change of at least about 60% of the normal cells. As an illustration of changes envisioned by the invention, for example, in the case of the N-CoR complex, lower levels of the complex are indicative of the presence of a disease or disorder. In contrast, for example, in the case of macrophages, VCAM1, MEKK1, TAB2, and the L/HX₇LL motif, higher levels are indicative of the presence of a disease or disorder. Also in the case of TAB2, a phosphorylated version of TAB2 is indicative of the presence of a disease or disorder.

As discussed above, those of skill in the art are well aware of, and fully capable of selecting and executing, appropriate techniques for detecting macrophages and other things of interest, which are indicative of a macrophage-induced signaling pathway. Examples of detection methods include, but are not limited to, immunohistochemical assays, Western blot analyses, ELISAs, RNA expression analyses, polyacrylamide gels, agarose gels, protein activity assays, PCR amplification, high-throughput gene expression analyses (such as micro array analyses), and detection of certain cell specific markers such as CD14, CD68, and CD16 for macrophages. Other detection methods are well known in the art and include methods that are not and need not be stated here.

In another aspect, the present invention has use in treating a disease or disorder, and thus provides a method of treating a disease or disorder. In general, the method comprises administering to a subject in need thereof at least one substance that affects the interaction with at least one macrophage with a molecule on the surface of another cell, wherein interaction of the macrophage with the other cell results in conversion of the cell to an abnormal cell (e.g., a cell of a disease or disorder). For example, the method can comprise administering a compound that inhibits the interaction of macrophages with one or more VCAM-1 molecules on the surface of other cells.

In a further aspect, the invention provides a method of treating a disease or disorder involving a macrophage-induced signaling pathway. In general, the method comprises administering to a subject in need thereof at least one substance that affects the expression or activity of a substance involved in a macrophage-induced signaling pathway. In preferred embodiments, the disease or disorder is one involving a sex hormone and/or a nuclear receptor or transcription factor for which activity is regulated, at least in part, by the presence of one or more sex hormones. Accordingly, in embodiments, the method is a method of treating a cancer relating to a sex hormone, such as prostate cancer and breast cancer. In yet other embodiments, the method is a method of treating a disease or disorder involving expression and/or activity of TAB2. In yet further embodiments, the method is a method of treating a disease or disorder involving expression and/or activity of MEKK1. In still further embodiments, the method is a method of treating a disease or disorder involving expression and/or activity of N-CoR or complexes comprising N-CoR.

In embodiments, the method of treating comprises administering to a subject at least one substance that affects the interaction between a macrophage-induced substance and expression of genes involved in a disease or disorder, where the substance(s) are administered in an amount sufficient to reduce or eliminate the disease or disorder. As with other methods of treating described herein, the act of administering may be repeated one or more times to achieve a desired effect. Thus, each dose or dosing of a substance, etc. need not provide a sufficient amount of the substance, etc. to achieve the desired goal, but rather the cumulative dosage may, in embodiments, provide the sufficient amount to achieve the desired goal.

The methods of the invention can involve administering any number of compounds, which can affect any number of interactions or physical activities of genes and gene expression products. However, typically the method involves administering a substance that affects the interaction of two or more proteins, either directly or via co-factors, that are involved in one or more signaling pathways between one or more cytokines and expression of a gene. In certain embodiments, the method is a method of improving the long-term activity of anti-cancer therapies by inhibiting the conversion of anti-cancer compounds to ineffective compounds or to cancer inducing compounds.

According to the methods of the invention, the subject, individual, or patient can be any organism to whom the treatment is administered. Thus, the subject may be a human or other mammal, including, but not limited to a rodent (e.g., mouse, rat, rabbit), a canine (e.g., a dog), a feline (e.g., a cat), an equine (e.g., a horse), an ovine (e.g., a sheep), an orcine (e.g., a pig), or a bovine (e.g., a cow or steer). The subject can be any other animal such as a bird, reptile, amphibian, or any other companion or agricultural animal. The invention thus has applicability in both human medical and animal veterinarian fields.

The methods of the invention generally comprise contacting at least one cell with at least one substance. The methods thus can be practiced in vitro, in vivo, and ex vivo. They accordingly may be practiced, for example, as a research method to identify compounds or to determine the effects of compounds and concentrations of compounds, as a therapeutic method of treating a disease or disorder involving a macrophage-induced signaling pathway, and as a method to prevent a disease or disorder. In embodiments where the method is a method of treating, it can be a method of therapy (e.g., a therapeutic method) in which the amount administered is an amount that is effective for reducing or eliminating a disease or disorder. In embodiments where the method is a method of prevention, the amount is an amount sufficient to prevent the disease or disorder from occurring or sufficient to reduce the severity of the disease or disorder if it does occur.

As used herein, a “sufficient amount” is an amount of a substance (e.g., a drug or compound) that produces a decrease in at least one detectable characteristic of a disease or disorder. For example, it may be a decrease in the size of a tumor, such as a cancerous tumor. It also may be a decrease in the rate of growth of a tumor. It further may be a reduction in the number of living cells of a tumor. It furthermore may be a reduction in the amount of disease-specific substances (e.g., cell surface molecules) being produced by a cancerous cell. Other characteristics can be immediately envisioned by those of skill in the art, and thus need not be listed herein. A sufficient amount of a substance can be administered in one or more administrations, in one or more doses. For purposes of this invention, a sufficient amount is an amount that produces a detectable change, preferably decrease, in transcription of a target gene, a detectable change, preferably decrease, in activity of a target protein, or a detectable change, preferably decrease, in interaction of a target protein with another substance or interaction of two or more target proteins (with each other or with co-factors or other small molecules that might affect three-dimensional structure or the ability of the protein to interact with other substances, such as other proteins or with nucleic acids). An amount sufficient to produce a detectable change, such as a detectable decrease, in these characteristics is typically an amount that provides a significant change in the levels of the characteristic of interest. In some instances, the change is a substantial change, such as a substantial decrease in gene transcription or protein activity.

The act of administering the substance(s) or compound(s) can be any act that provides the substances or compounds to the body of the subject so that they can function for their intended purpose. The substances, etc. can be administered by any suitable route, in any suitable amount, and by way of any suitable regimen. Thus, the substances can be, for example, administered orally, as a pill, capsule, caplet, powder, liquid, gel, salve, cream, lozenge, tablet, or any other suitable oral delivery vehicle. The substances can also be delivered, for example, in an injectable or infusible form, such as a liquid suitable for intravenous injection or infusion or injection directly into a body site, such as a neoplastic site (e.g., site of cancer or tumor growth), subcutaneously, and intramuscularly. Alternatively, the substances can be formulated, for example, to be absorbed via mucosal membranes or skin, and thus can be in a salve, cream, gel, or the like for topical, intranasal, sublingual, intrarectal, or intravaginal delivery. Amounts to be administered will vary depending on the administration route. One of skill in the art is capable of determining the appropriate amount of substance (e.g., anti-cancer agent) to be administered to a subject in need based on well-understood principles of pharmacology. The amounts and number and frequency of repetitions of administrations may be adjusted, according to well known principles of medicine, after one or more administrations or doses, in consideration of both the beneficial effects and deleterious effects (e.g., side-effects) of the substance(s) on the patient.

Furthermore, cancer treatment regimens are well known in the art, and any suitable regimen may be used to treat the cancers encompassed by this invention. Thus, treatment regimens may comprise, among other things, single doses of one or more substance, or multiple doses of one or more substances. Dosing may be repeated at regular intervals or only as deemed necessary. Likewise, multiple different administrations and routes may be used, based on response to initial dose(s) and the type progression of the cancer. Those of skill in the art are well aware of the numerous dosing regimens available, and thus the various regimens need not be disclosed here. One of skill in the art may select the appropriate dosing regimen based on various well understood factors without undue experimentation. Of course, the method of treating can be implemented as the sole therapy for a cancer, or can be used in conjunction with other modes of treatment, as part of a multiple mode treatment regimen. Treatment for other diseases and disorders encompassed by the invention are likewise amenable to well-known regimens, and thus the details for those regimens, and any modifications, need not be detailed herein.

In one embodiment of a method to treat a disease or disorder, the compound to administer is an inhibitor of MEKK1 activity or expression. The present invention shows that there is a requirement for MEKK1 in abolition of efficacy of cancer agonists, or in conversion of cancer antagonists to cancer agonists. In particular, MEKK1 is an enzyme that phosphorylates TAB2, resulting in activation of transcription and progression to an abnormal disease or disorder state. Thus, the invention contemplates use of an inhibitor of MEKK1-TAB2 interaction. It also contemplates the use of an inhibitor that binds to MEKK1 and reduces or abolishes its TAB2 phosphorylation activity. Such an inhibitor can be a simple, small organic or inorganic molecules, such as a secondary metabolite, a metal, or another small molecule that can pass through biological membranes. Alternatively, such an inhibitor can be a more complex molecule that is natural or synthetic, such as a peptide, polypeptide, or protein, which may be modified by inclusion of moieties that improve one or more characteristics of the molecule (such as solubility, membrane permeability, stability/resistance to degradation, and the like). Such modifications are well known in the biochemistry field, and need not be detailed herein.

As mentioned, the inhibitor can be an inhibitor of gene transcription, and lead to the production of less MEKK1 mRNA. It thus may be an inhibitor of one or more transcription factors or one or more polymerases (or portions thereof). It likewise may be a DNA intercalating agent, including, but not limited to, any of the known intercalating agents used in the art. Preferably, the inhibitor is specific for the MEKK1 gene, such as, for example, a nucleic acid that binds specifically to MEKK1 gene sequences and interrupts transcription. In addition, the inhibitor can function at the level of translation and cause less MEKK1 protein to be produced. For example, the inhibitor may be an inhibitor of ribosomes. Preferably, the inhibitor is specific for MEKK1 mRNA, such as, for example, a nucleic acid that binds specifically to MEKK1 mRNA sequences (e.g., a small interfering RNA (siRNA) for RNA interference, or an antisense molecule). Such MEKK1 gene and mRNA specific molecules may easily be designed and implemented by those of skill in the art, and thus specific examples need not be disclosed herein. The inhibitor can also function at the level of protein activity and block at least one MEKK1 function, preferably the ability to interact with TAB2. Thus, treating with an inhibitor of MEKK1 is an anti-cancer treatment according to the invention. The inhibitor can be, for example, an antibody or any other inhibitor that blocks expression or activity of MEKK1. This method includes a substance that prevents binding of MEKK1 to another molecule involved in this signaling pathway. As should be evident, the invention thus provides a method of treating cancers, such as prostate and breast cancers, with one or more inhibitors of MEKK1 expression or activity.

Another method can include administering an inhibitor of VCAM1 expression or activity, such as VCAM1 interaction with a macrophage. VCAM1 is involved in attraction and/or adhesion of macrophages to cells, such as cancer cells. The present invention discloses that substances produced by or in response to the presence of macrophages, such as cytokines, abolish the activity of repressors of cancer, such as SARMs and SERMs. By inhibiting VCAM1 expression or the ability to interact with macrophages, cancer cells and other disease or disorder cells will not attract and adhere or interact with macrophages to an appreciably greater extent than normal, and development of cancers, or at least a resistance to anti-cancer agents, will be inhibited. Likewise, development of other diseases and disorders involving macrophage induction of a signaling pathway, and resistance to agents that treat such diseases and disorders, will be inhibited. As with the inhibitor of MEKK1 expression and activity, the inhibitor for VCAM1 can be at the level of gene transcription, translation, or activity of the protein. It thus can be, among other things, a nucleic acid that specifically interferes with DNA transcription, mRNA stability, or mRNA translation. It also may be, among other things, an antibody or any other inhibitor that blocks expression or activity of the VCAM1 protein. As with the inhibitors for MEKK1, design and production of VCAM1 inhibitors can be accomplished by those of skill in the art without undue or excessive experimentation, and thus details regarding such design and production need not be detailed herein.

In a particular embodiment, the method comprises administering a substance that interferes with the interaction between VCAM1 and macrophages. Blocking the interaction of VCAM1 and macrophages reduces the production of macrophage-derived substances (and in particular, one or more cytokines) in proximity to the cells expressing the VCAM1 (e.g., cancer cells, such as prostate and breast cancer cells), and reduces or inhibits the conversion of cells to cancerous cells, reduces or inhibits the development of cancer cells, and reduces or inhibits the development of resistance to anti-cancer agents in cancer cells. It likewise has a similar effect on the development and progression of other cells that express VCAM1 and can be converted to abnormal cells as a result of interaction with macrophages. As should be evident, the invention thus provides a method of treating cancers, such as prostate and breast cancers, with one or more inhibitors of VCAM1 expression or activity. In embodiments, it is a method of treating cancers, such as prostate and breast cancers, with one or more inhibitors of VCAM1 interaction with a macrophage.

Yet another method can include administering a substance that inhibits TAB2 expression or activity. The effects of macrophage-derived substances on development of cancers and development of resistance to anti-cancer substances are shown herein to rely, at least in part, on the presence of TAB2 in a complex with other proteins, including hormone receptors. Thus, inhibiting TAB2 expression or ability to participate in such complexes abolishes or reduces the effect of macrophage-derived substances on the development of cancer and resistance to anti-cancer agents. Similarly, inhibiting TAB2 expression or ability to participate in such complexes abolishes or reduces the effect of macrophage-derived substances on the development of other diseases and disorders involving the macrophage-induced signaling pathway discussed herein, and any resistance that might develop to agents used to treat such diseases and disorders. As with the inhibitor of MEKK1 and/or VCAM1 expression and activity, the inhibitor for TAB2 can be at the level of gene transcription, translation, or activity of the protein. It thus can be, among other things, a nucleic acid that specifically interferes with DNA transcription, mRNA stability, or mRNA translation. It also may be, among other things, an antibody or any other inhibitor that blocks expression or activity of the TAB2 protein. As with the inhibitors for MEKK1 and VCAM1, design and production of TAB2 inhibitors can be accomplished by those of skill in the art without undue or excessive experimentation, and thus details regarding such design and production need not be detailed herein. Non-limiting examples of inhibitors include those similar to ones specific for MEKK1 and/or VCAM1, i.e., TAB2-specific antisense nucleic acids, TAB2-specific siRNA molecules, and TAB2-specific antibodies (monoclonal or polyclonal).

In embodiments, the method comprises administering a substance that interferes with phosphorylation of TAB2. The conformational change upon phosphorylation of TAB2 enhances its association with the N-CoR complex, which enables its role for dismissal of the entire N-CoR “holocorepressor” complex and activation of transcription. Thus, blocking phosphorylation of TAB2, and subsequent association with the complex abolishes or reduces the effect of macrophage-derived substances on the development of cancer and other diseases and disorders. Non-limiting examples of ways to block phosphorylation include, but are not limited to, administering an inhibitor of TAB2 phosphorylation or administering a peptide, polypeptide, or protein having a sequence identical or similar to TAB2, but mutated to destroy the phosphorylation site. The sequences for such molecules are known in the art and can easily be found or derived from sequences in public databases (e.g., MEKK1, VCAM1, and TAB2). Thus, the various sequences need not be detailed herein. For example, the active site of the MEKK1 enzyme (also known in the art as mitogen-activated protein kinase kinase kinase 7-interacting protein 2) is the kinase domain of the protein, which comprises an active site lysine residue (the lysine residue (residue 1255 of the overall sequence) in the human sequence: YQAQDVGTGTLMAVKQVTYV), which has been shown to be involved in enzymatic activity through its binding to ATP. It should be recognized that many kinase inhibitors are ATP analogs. Furthermore, in the case of the transmembrane VCAM1 protein, the active site is the entire extracellular domain. In the case of TAB2 (also known in the art as TAK1-binding protein 2), the active site of the human protein is know (see, for example, Baek et al., Cell, 2002), and comprises the sequence TNSGASAASRNMSGQVSMBPAFIHHHPPKS. At the active site, functional studies have shown that alanine mutants do not have the same biological activity as the wild-type, and that the two serine residues in bold (S419 and S423) are residues that comprise the site that is relevant to antagonist function.

Alternatively or in addition, the method can comprise administering a substance that changes the expression or activity of an N-CoR complex. N-CoR is a multi-component complex that prevents transcription of genes. As discussed above, complexes of hormone receptors and N-CoR and TAB2 are affected by substances derived from macrophages, such as cytokines. The effect of the cytokines, such as IL1-β, on the complexes is to convert the complexes from transcription antagonists (i.e., to keep transcription turned off) to transcription agonists (i.e., allowing transcription). Binding of TAB2 to N-CoR is involved in resistance of the complexes to receptor modulators, such as SARMs and SERMs. Accordingly, the invention provides a method of administering a substance that modifies the activity or expression of the N-CoR complex such that the complex is not converted from an antagonist to an agonist. As non-limiting examples, this method can involve use of a substance that blocks binding of TAB2 to the N-CoR complex or a substance that prevents the dismissal of the complex from the gene of interest. In embodiments, the substance is a peptide comprising the amino acid motif L/HX₇LL. In embodiments, such substances would include a peptide comprising the amino acid motif L/HX₇LL.

The method of treating cancer or other diseases or disorders can thus comprise administering one or more substances that interfere with the binding of TAB2 to an antagonist/N-CoR complex. For example, the method can comprise administering a poly-amino acid comprising the L/HX₇LL motif sequence, or a functional equivalent of it (e.g., peptidomimetic), to a subject to affect binding of TAB2 to a complex comprising the N-CoR complex. In practicing the method according to this embodiment, the poly-amino acid comprising the L/HX₇LL motif can act as a competitor of a hormone receptor for TAB2, and thus reduce binding of TAB2 to the receptor and consequential increases in transcription. The effects of macrophage-derived substances on development of cancers and development of resistance to anti-cancer substances are shown herein to rely, at least in part, on the presence of TAB2 in a complex with other proteins including hormone receptors. Thus, blocking or reducing binding of TAB2 to the N-CoR complex, for example by way of interaction of TAB2 with a hormone receptor on a nuclear membrane, will prevent or reduce the ability of the complex to be converted from an antagonist to an agonist. Peptidomimetics according to the invention include all poly-amino acid molecules that comprise the L/HX₇LL motif or its functional equivalent. In some embodiments, the peptidomimetic is a peptide, polypeptide, or protein comprising the L/HX₇LL motif and an unlimited number of additional amino acids at one or both ends of the motif, where the additional amino acids do not block access of TAB2 to the motif. In other embodiments, the L/HX₇LL motif comprises an amino acid sequence that is not identical to the motif, but provides the same three-dimensional structure as seen in the nuclear receptors from which it is derived. Thus, for example, the motif may be LX₇LL, HX₇LL, IX₇LL, or other similar motifs. Conservative substitutions (according to widely recognized parameters) for one or more of the leucines is contemplated, although substitution of one or more leucines with alanine is not preferred. The spacing between the leucines is preferred to be seven amino acids, but a spacing of six or eight is acceptable. Changes in spacing are particularly contemplated when non-standard linkages between amino acids are used, for example when the non-standard linkages cause a lengthening or shortening of the typical peptide bond or cause the rotation of amino acids about bonds to change.

In another embodiment, the method of treating cancer or another disease or disorder can comprise administering one or more compounds that counteract the effect of one or more cytokines, such as IL-1β, on a hormone receptor. The method can comprise administering a substance that directly binds the cytokine, such as an antibody that binds IL-1β. Alternatively, it can be a substance that blocks binding of the cytokine to a substance that is required for derepression of gene expression of a hormone receptor-regulated gene. For example, it can be an antibody that blocks binding of IL-1β to a complex comprising the androgen receptor by binding to and blocking the binding site of IL-1β on the complex. In embodiments, the method of this embodiment can comprise administering at least one compound that binds NcoR but does not activate expression of genes regulated by complexes in which NcoR is found.

Further, a method according to the invention can comprise administering a substance that binds to an L/HX₇LL motif, but does not activate expression of genes regulated by complexes comprising one or more proteins having that motif as part of its amino acid sequence. The present invention discloses that L/HX₇LL motifs are involved in recruitment of TAB2 to complexes comprising hormone receptors, such as androgen receptors and estrogen receptors, and that such recruitment is necessary for conversion of antagonists of gene expression via the receptors (e.g., the antagonist function of SARMs) to agonists of gene expression. Accordingly, substances that bind to the L/HX₇LL motif without activating the complex will block or reduce the binding of TAB2 to complexes, and block or reduce the conversion of such complexes to agonists of gene activity.

In addition or alternatively, the method can comprise administering a nucleic acid comprising a nucleotide sequence that encodes one or more L/HX₇LL motifs, or peptidomimetics, to a subject, and permitting the nucleic acid to be expressed in at least one cancer or diseased tissue cell, to reduce or block development or continuance of that cell as a cancer or diseased cell. In such a method, which can be a method of gene therapy, the nucleic acid can be incorporated in a cellular chromosome as a permanent part of the genome, or can be maintained in the cell as an extra-chromosomal element for a transient effect, such as to reduce proliferation of the cell while other anti-cancer modalities are being employed. As with administering a peptide comprising this motif, or a peptidomimetic (as discussed above), expression of numerous copies of the motif can act to titrate out TAB2 molecules, reducing or eliminating binding of TAB2 to the motifs present on one or more proteins involved in complexes with hormone receptors.

In accordance with the discussion above, the method can comprise administering a substance that interferes with the interaction between macrophages and cells of a disease or disorder. The invention discloses that it is the interaction between macrophages and cells that activate the signaling pathway leading to a change in gene transcription. Blocking the interaction between the macrophages and cells of a disease or disorder is a way to reduce or eliminate the disease or disorder. One example of this is an antibody that binds to a site on the macrophage or to a binding site on the cell (e.g., VCAM1), which prevents interactions between the macrophage and the cell.

In embodiments, the disease or disorder to be treated is a cancer that shows altered gene expression in the presence of one or more hormone receptors, or as a result of the activity of one or more hormone receptors. Thus, in embodiments, the cancer is prostate cancer. In other embodiments, the cancer is breast cancer. In embodiments, the method affects androgen receptor mediated expression of genes. In other embodiments, the method affects estrogen receptor mediated expression of genes. In yet other embodiments, the method affects progesterone mediated expression of genes.

In another aspect, the invention provides methods of screening for substances that are effective at treating at least one disease or disorder. In general, the methods comprise contacting at least one gene or gene expression product with a substance, and determining if the substance affects the expression of the gene or the activity of the gene expression product. In certain embodiments, the method determines if the level of expression or activity is reduced or eliminated. In other embodiments, the method determines if the level of expression or activity is increased. In yet other embodiments, the method can be a two step or greater method that incorporates several of the screening methods discussed herein. The method has use in screening for substances that have use in treating at least one disease or disorder.

The method of screening can be a method of screening for substances (e.g., drugs, lead compounds, pharmaceutical agents, etc.) to treat a disease or disorder, where the method comprises using an MEKK1 expression or activity inhibition assay. In these embodiments, the method can comprise contacting at least one molecule of MEKK1 or a nucleic acid encoding MEKK1 with a substance, performing a MEKK1 expression or activity inhibition assay, and determining the MEKK1 levels of expression or activity. In embodiments, the contacting is accomplished simply by placing the two substances in an environment that is amenable to their contact, and providing sufficient time for contact to occur. The invention discloses that MEKK1 is important in reduction or abolition of efficacy of cancer agonists, or in conversion of cancer antagonists to cancer agonists. Thus, screening for an inhibitor of MEKK1 expression or activity, whether it be an inhibitor of protein function or an inhibitor of mRNA or protein expression, can be a method of screening according to the invention. In one non-limiting example, the method comprises screening using a MEKK1 binding assay. Numerous inhibition assays are known in the art, including but not limited to those disclosed herein, and any suitable assay can be used in accordance with the invention.

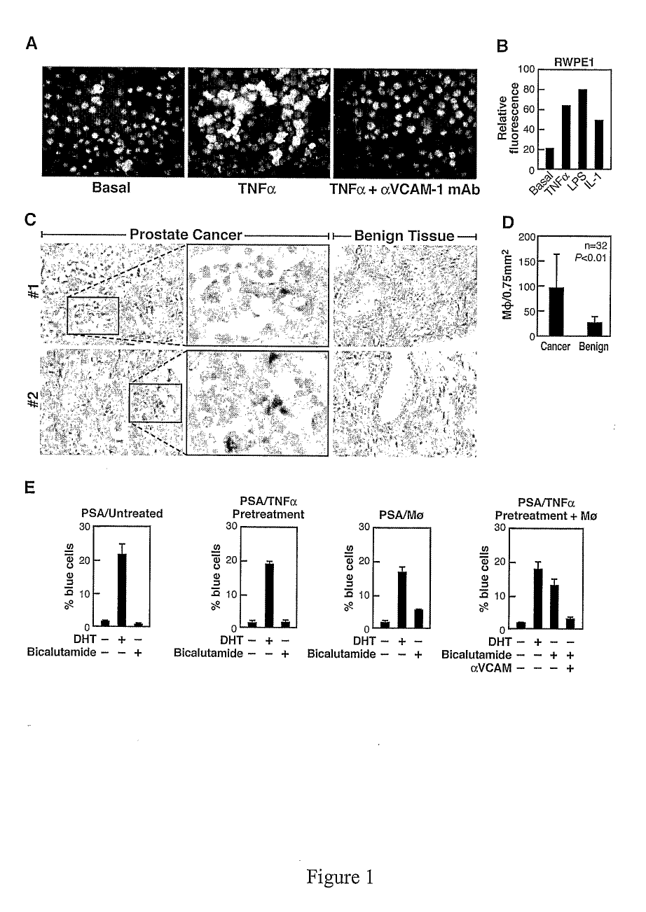

In other embodiments, the method of screening can be a method of screening for new agents to treat a disease or disorder using a VCAM1 inhibition assay. Like the MEKK1 assays discussed above, these assays can be assays for inhibition of gene expression, protein expression, or inhibition of protein activity. The method can comprise contacting at least one molecule with a VCAM1 inhibition assay and determining the VCAM1 levels of expression, activity or binding. In addition, like the MEKK1 assays, numerous inhibition assays are known in the art, including but not limited to those disclosed herein, and any suitable assay can be used in accordance with the invention. The invention discloses that VCAM1 is involved in attraction and/or adhesion of macrophages to cells, such as cancer cells. Interruption of this interaction by inhibition or abolition of gene expression or by inhibition or abolition of VCAM1 activity in interacting with macrophages can identify substances that would function in vivo as anti-cancer agents. In one embodiment, the assay can be a VCAM1/macrophage interaction inhibition assay. This approach has been successful to provide proof of principle, employing fluorescence-labeled macrophages and prostate tumor cells (see, for example, FIG. 1, panels A and B, below). This quantitative assay can be used to screen drug libraries, or to evaluate the effectiveness of newly developed molecules, based upon their ability to block the interaction of VCAM1 with macrophages.

According to another embodiment of the method of screening, the method can be a method of screening for new agents to treat a disease or disorder using a TAB2 inhibition assay. The method can comprise exposing at least one molecule of TAB2 to a substance, performing a TAB2 inhibition assay, and determining the TAB2 levels of expression, activity, binding, or phosphorylation. In embodiments, the method is a method of inhibiting or detecting inhibition of expression of TAB2, while in others it is a method of inhibiting or detecting inhibition of binding of TAB2 to complexes comprising a hormone receptor. It thus can be a method of screening for substances that block the ability of TAB2 to participate in derepression of gene expression of genes regulated by hormone receptors, such as androgen receptors and estrogen receptors. The invention discloses that binding of TAB2 to complexes comprising hormone receptors is important not only in repression of expression of certain genes, but is also important in macrophage-induced resistance to anti-cancer substances. The macrophage-induced resistance relies, at least in part, on the presence of TAB2 in the complex. Thus, assays that utilize detecting of binding of TAB2 to complexes, or that utilize detecting expression of TAB2, can be used as assays for substances that identify anti-cancer agents. MEKK1-dependent phosphorylation of TAB2 enhances its interactions with the N-CoR complex, consistent with its function in removal of the N-CoR holocorepressor complex in derepression. Assays that detect phosphorylation of TAB2 can also be used as assays for substances that identify anti-cancer agents and agents that can be used to treat other diseases and disorders involving a macrophage-induced signaling pathway. Thus, in embodiments, the invention provides for the use of phospho-specific antibodies for the purpose of detecting and evaluating TAB2 phosphorylation in cells and tissue samples. The use of such antibodies, and the results generated, provide both diagnostic and prognostic screening methods, based on the knowledge that increased TAB2 phosphorylation in a tumor cell is predictive of hormone resistance. Use in screening methods according to the invention are thus provided.

Alternative or additional methods rely on inhibition of activity of N-CoR. These methods can comprise contacting at least one N-CoR complex to a substance, performing a N-CoR inhibition assay, and determining the N-CoR levels of expression, activity, or binding. As discussed above, complexes of hormone receptors and N-CoR and TAB2 are affected by substances derived from macrophages, such as cytokines. The effect of the cytokines, such as IL1-β, on the complexes is to convert the complexes from transcription antagonists to transcription agonists. Binding of TAB2 to N-CoR appears to be required for resistance of the complexes to receptor modulators, such as SARMs and SERMs. Accordingly, assays that identify inhibitors of the activity of N-CoR, particularly those that affect binding of N-CoR to TAB2, to a macrophage-derived substance or substance produced in response to macrophage interaction with a cell, or to an agonist or antagonist of a hormone receptor, can be used to identify substances that can affect the process of cancer formation or progression. Preferably, the identified substance does not have the same or similar activity as the TAB2, cytokine, etc.

In yet another embodiment of the method of screening, TAB2 is used to identify substances that block interaction of a macrophage-derived substance, such as a cytokine, to a complex comprising TAB2, yet does not mimic the function of the macrophage-derived substance on the activity of a hormone receptor. Numerous analogs of macrophage-derived substances, such as analogs of cytokines, can be screened for their ability to block binding by macrophage-derived substances, yet at the same time not convert a complex of N-CoR, TAB2, and/or hormone receptor from one that antagonizes gene expression to one that agonizes it.

In another embodiment of the screening method, a peptide (or nucleic acid encoding it) comprising the motif L/HX₇LL or a peptidomimetic of this motif is used to identify substances that block binding of TAB2 to a complex comprising a hormone receptor. The present invention discloses that this motif is important in binding of TAB2 to such complexes, and that such binding is important in the effects of macrophage-derived substances on resistance to anti-cancer compounds. By using the motif, as a single motif or in conjunction with other multiple copies of it, one can identify substances that can block binding of TAB2, and thus eliminate the effects of macrophage-derived substances on complexes having a protein with that motif.

In another embodiment, the method provides a screening method that utilizes counteracting the effect of one or more cytokines, such as IL-1β, on a hormone receptor. This embodiment of the method can comprise contacting at least one molecule of a cytokine to a substance, performing a cytokine inhibition assay, and determining the levels of cytokine expression, activity, or binding. As with all of the other methods, the act of contacting may be direct or indirect, and may rely on human-induced or natural phenomena, such as diffusion, brownian motion, shuttling, mixing, stirring, and the like. This embodiment of the method can be used to screen for substances that directly bind the cytokine, such as an antibody, or for substances that block binding of the cytokine to a substance that is required for derepression of gene expression of a hormone receptor-regulated gene.

The present invention also provides a method of screening for substances that inhibit, reduce, or abolish the effects of macrophage-derived or macrophage-induced substances on expression of genes controlled by hormone receptors. In these methods, N-CoR−/− cells are used. The cells may be used in culture (i.e., in vitro) or may be used in an in vivo model (i.e., a knock-out animal, such as a knock-out mouse, rat, rabbit, etc.). The method comprises contacting at least one N-CoR −/− cell with at least one substance, and determining the levels of expression of genes involved in a macrophage induced signaling pathway.

As it is disclosed herein that macrophage-derived or induced substances are involved in the resistance of certain cancers to anti-cancer substances, the invention provides methods of screening for substances that prevent, delay, inhibit, or lessen the onset of resistance to these anti-cancer substances. The methods utilize the same method steps discussed above, and are generally based on inhibition assays known in the art.

Furthermore, in view of the mechanism of action discussed herein, the invention provides methods for identifying substances that affect the interaction of diseased cells with macrophages. Because the methods can identify a characteristic of diseased cells that is different than methods that rely on cell morphology or surface markers, it can be used not only as a stand-alone method for identifying diseases or disorders, but as a confirmatory assay to supplement other assays. Furthermore, because it can be quickly and inexpensively performed using standard laboratory equipment, it might have advantages for use in certain settings over other methods, which might be more costly or rely on expensive reagents and/or equipment.

In general, the methods of screening of the invention can be practiced on single substances or compounds, or on collections of substances or compounds. The substances or compounds may be present in the assay compositions as known entities and/or in known amounts, or may be present as unknown substances and/or in unknown amounts. Where multiple substances are screened in one execution of the method, samples showing a desired result may be re-screened or the substances present in the sample fractionated, for example by dividing the constituent substances into multiple successive samples having fewer substances per sample, and re-screened. This iterative process may be repeated until a single, or multiple single, substances are identified. The methods thus may be methods of high-throughput screening of known or unknown compounds. They likewise may comprise identifying one or more compound or substance of interest. Techniques for identifying chemical, biochemical, and inorganic substances are well known in the respective arts, and thus need not be detailed herein. Of course, as mentioned above, the method may further comprise performing one or more positive or negative controls to assist in the selection of desired substances or in identification of a substance.

In yet another aspect, the invention provides methods to affect, for example by increasing or decreasing, fertility. The methods rely on using anti-antigens with TAB2. More specifically, the invention discloses that the mechanisms involved in hormone receptor-mediated gene expression are conserved, at least to some extent, in reproductive biology. The mechanism is active at the implantation stage of a blastocyst in the uterine lining. Blastocyst implantation involves a local induction of BMP7 in response to inflammatory signals. By regulating the effects of cytokines on the blastocyst and maternal cells, implantation can be enhanced or reduced, resulting in an increase or decrease in rate of successful impregnation. The method of this aspect of the invention comprises administering a substance to a subject that changes the expression of a gene involved in blastocyst implantation. Typically, the subject is a female. In one non-limiting example, the method comprises administering an antibody that specifically binds TAB2 to a subject that causes an increase in blastocyst implantation. The method has use in treating subjects for infertility or reduced fertility or as a way to increase fertility. Administration can be by any suitable route, including, but not limited to, intrauteral.

In a further aspect, the invention provides substances discovered using the methods of the invention. In general, the substances are drugs that can be used to treat a disease or disorder, such as a cancer or resistance to anti-cancer agents, diabetes or insulin resistance, and atherosclerosis or resistance to anti-atherosclerosis agents. These can comprise inhibitors, analogs, antibodies, mutated versions of proteins or nucleic acids, antisense oligomers, or any other type of drug known in the art. While many of the drugs may have been known in the art previously, the knowledge provided by the present invention permits reformulation of those drugs into novel compositions that are highly effective in treating diseases or disorders involving expression of genes under the control of hormone receptors.

In another aspect, compositions comprising substances that cause a change in macrophage-induced transcription of at least one gene, or in the activity of at least one protein involved in a macrophage-induced signaling pathway are provided. In general, a composition of the invention comprises a sufficient amount of at least one substance to cause a detectable change in transcription of at least one gene, or in the activity of at least one gene product, where the gene or gene product is involved or regulated by a macrophage-induced signaling pathway. Preferably, the pathway involves TAB2, MEKK1, and/or a complex comprising N-CoR. Thus, when used in methods of treating, the compositions comprise at least one substance in an amount effective to cause a detectable change in macrophage-induced transcription of at least one gene in a cell of a subject. In embodiments, the substances are provided in an amount sufficient to provide one or more doses to a subject. In embodiments relating to in vitro use of the substances, the amounts can vary widely, but generally are sufficient to perform at least one assay for the effect of the substance on gene expression or protein expression or protein activity. Certain other aspects of the invention provide for use in the preparation of compositions for medical use, such as pharmaceutical or therapeutic compositions. Compositions may comprise substances of the invention along with one or more other substances, which are typically substances that are biologically tolerable in that they may be exposed to living cells without killing the cells. In embodiments, the compositions can comprise cells, tissues, proteins, nucleic acids, or other small or complex molecules typically found in biological samples. Compositions may also comprise some or all of the reagents, compounds, labels, etc. that are used in one or more of the various assays mentioned herein. In embodiments, the compositions comprise one or more substances that are pharmaceutically acceptable. As used herein, a “pharmaceutically acceptable substance” is one that is not toxic to a cell to which it is contacted, at the concentration at which it is contacted with the cell. The pharmaceutically acceptable substance thus may be toxic at the level present in the composition, but upon administration to a subject, is diluted to a safe level. The term is thus intended to include, but not be limited to, solvents, coatings, antibacterial and antifungal agents, and any other ingredient that is biologically tolerable. Examples of such substances include, but are not limited to, water, saline, human serum albumin, sugars, salts, lipids, drugs, carriers, flavorants, fillers, binders, gums, colorants, buffers, detergents, biologically active compounds, and the like. The use of such pharmaceutically active substances is well known in the art. Preferably, and particularly for compositions intended for in vivo use, the composition is sterile or has been sterilized. Sterilization may be performed by any suitable technique.

Another aspect of the invention provides a container comprising one or more substances that cause a change, such as a decrease, in the macrophage-induced transcription of genes. In general, where designed for in vivo treatment of a subject, a container according to the invention contains a sufficient amount of substance to provide at least one dose to the subject. Where designed for in vitro use, a container typically contains at least enough of at least one substance to conduct an assay according to the invention. In certain embodiments, the container is provided in a package with one or more other containers and/or with one or more articles of manufacture or devices having use in delivery of substances to subjects (e.g., syringes, needles, antiseptic swabs), or for practice of the methods of the invention in vitro.

The present invention also provides kits. In general, the kits comprise a sufficient amount of at least one substance to cause a detectable change in transcription of at least one gene. Typically, the substance will be supplied in one or more container, each container containing a sufficient amount of substance for at least one dosing of the patient. The kits can comprise other components, such as some or all of the components necessary to practice a method of the invention. The kits may contain a syringe for administering a dose of the substance. The kits may also comprise filters for sterilization prior to delivery. They may likewise contain sterile water or buffer for rehydration or reconstitution of dry substance, prior to administration to a patient. In embodiments, multiple doses of substance are provided in the kit, either all in a single container (e.g., a vial) or distributed among two or more containers. Preferably, the kit and its contents are sterile or have been sterilized.

### EXAMPLES

The invention will be further explained by the following Examples, which are intended to be purely exemplary of the invention, and should not be considered as limiting the invention in any way.

### Example 1

### Materials and Reagents

The detailed exemplary disclosure that follows, and particularly the experiments and data, were based on assays and experiments that are disclosed in this Example, unless otherwise noted.

Antibodies

The following antibodies were obtained from Santa Cruz Biotechnology: anti-AR, ERα, HDAC1, HDAC2, HDAC3, MBD3, MEKK1, MEK1, CBP, p300, p/CAF, SRC1, p/CIP, GRIP1, PBP, BRG1, and mSin3A/B. The following commercially available antibodies were used: anti-Tip60, CARM1, acetylated histone H3, acetylated histone H4, dimethyl-histone H3 (R17), dimethyl-histone H3 (R26), and dimethyl-histone H4 (R3) (Upstate Biotechnology), anti-RNA Polymerase II antibodies (Berkeley Antibody Company), anti-TAB2 (Affinity BioReagents), anti-HA (Covance), anti-Flag (Sigma). Anti-N-CoR and TBLR1 antibodies were described (Perissi et al., 2004).

Substrates and Concentrations/Dosages

Fluorogenic histone deacetylase substrate (Boc-Lys(Ac)-AMC) and human IL-1β were from Calbiochem. Dihydrotestosterone (DHT), cyproterone acetate (CPA), 17-β-Estrodial (E2) and 4-OH tamoxifen (4-OHT) were purchased from Sigma. The concentrations of treatment were: IL-1β (10 ng/ml), DHT (20 nM), CPA (1 uM), E2 (10-20 nM), and 4-OHT (1 uM) if not indicated. The Fugene 6 reagent (Roche) and Lipofectamine 2000 (Invitrogen) were used for transfection experiments following the manufacturers' manuals.

Small Interfering RNA (siRNA)

Sequences for siRNA for N-CoR were as follows: sense strand: 5′-GCACCGAAGUAUUGUCCAAdTdT-3′ (SEQ ID NO:1); antisense strand: 5′-UUGGACAAUACUUCGG UGCdTdT-3′ (SEQ ID NO:2). The siRNA pool for HDAC1, HDAC3 (SMARTpool HDAC1 and HDAC3), and non-specific control were purchased from Dharmacon. The siRNA targeting TAB2 (sc-41049) was obtained from Santa Cruz Biotechnology.

Indirect Immunofluorescence Analysis and Deconvolution Microscopy

Cells were grown on coverslips and washed with PBS three times. Cells were then fixed in 2% paraformaldehyde in PBS for 15 min at 37° C., washed in PBS, and permeabilized with 0.02% Triton X-100 in PBS for 30 min at room temperature. Blocking was performed with 0.1% BSA, 0.1% gelatin, 0.1% preimmune-serum in PBS for 30 min. For staining, cells were incubated with affinity-purified anti-N-CoR IgG overnight, followed by three washes in PBS. Stained cells were incubated for 2 hours with Alexa Fluor 546-conjugated secondary antibodies (Molecular Probes), followed by three washes in PBS. Fluorescent slides were imaged using deconvolution microscopy (University of California, San Diego).

Single Cell Nuclear Microinjection Assays and Luciferase Reporter Assays

Microinjection assays were carried out as previously described (Kamei et al., 1996; Jepsen et al., 2000). Each experiment was performed on three independent cover slips consisting of 1,000 cells, with >300 cells injected. Where no experimental antibody was used, preimmune rabbit or guinea pig IgG was coinjected, allowing the unambiguous identification of injected cells in addition to serving as a preimmune control. Antibodies to HDACs are as previously described (Jepsen et al., 2000). Single cell nuclear microinjection-coupled RNA expression profiling was performed as described previously (Perissi et al., 2004). The 14mer peptides (DILSEASTMQLLQQ (SEQ ID NO:3 and DIASEASTMQAAQQ (SEQ ID NO:4)) were synthesized and purchased from Sigma-Genosys. The luciferase reporter assays were carried out by co-transfection of the ERα expression plasmids, a reporter driven by the human BMP7 proximal promoter and a β-Gal expression plasmid as the internal control. Cells were then cultured in the charcoal-stripped medium for 48 hours and finally treated with compounds for 12 hours. The luciferase activities were normalized by the corresponding β-galactosidase activities.

Chromatin Immunoprecipitation (ChIP) Assays

The CHIP assay was conducted as previously described (Jepsen et al., 2000; Shang et al., 2000; Zhu et al., 2004), with average size of sheared fragments about 300-500 bps. For PCR, 1-5 ul from 50 ul DNA extraction and 25-35 cycles of amplification were used. The primer sequences were as follows:

| | |
|---|---|
| KLK2-5′ AGCCTTCATTCTCCAGGACC; | (SEQ ID NO:5) |
| KLK2-3′ CGTGAGAATGCCTCCAGACT; | (SEQ ID NO:6) |
| PSA-5′ AGGGATCAGGGAGTCTCACA; | (SEQ ID NO:7) |
| PSA-3′ GCTAGCACTTGCTGTTCTGC; | (SEQ ID NO:8) |
| BMP7-#1-5′ TCTCTGGGAGGAGAAAGCAG; | (SEQ ID NO:9) |
| BMP7-#1-3′ TTGCCCTCAGTCCTGTATCC; | (SEQ ID NO:10) |
| BMP7-#2-5′ CGCTATCAGTCACCCCATTT; | (SEQ ID NO:11) |
| BMP7-#2-3′ CGAAAAGGCTTTGAGATTGC. | (SEQ ID NO:12) |

The primers for RARb promoter were previously described (Perissi et al., 2004).

Tissue Microarray and Immunohistochemistry

Prostate tissue microarrays (Cat# A302) was purchased from ISU Abxis (Seoul, South Korea) containing prostate cancer tissues with corresponding normal tissues. For detecting infiltrating macrophages in prostate tissues, anti-CD68 antibody (Clone KP-1, Oncogene, Cat # OP172, 1:50 dilution) was used for staining. Staining was performed as previously described (Zhu et al., 2004).

RT-PCR

Semi-quantitative RT-PCR was carried out as described (Zhu et al, 2004). Primers were:

| | |
|---|---|
| BMP7, | |
| 5′-TCGTGGAACATGACAAGGAA-3′ | (SEQ ID NO:13) |
| and | |
| 5′-CTGATCCGGAACGTCTCATT-3′; | (SEQ ID NO:14) |
| MKNK2, | |
| 5′-TGGAGATGCTGTACCAGTGC-3′ | (SEQ ID NO:15) |
| and | |
| 5′-AGAGGATGTTTTCCGGCTTT-3′; | (SEQ ID NO:16) |
| pS2, | |
| 5′-TTTGGAGCAGAGAGGAGGCAATGG-3′ | (SEQ ID NO:17) |
| and | |
| 5′-TGGTATTAGGATAGAAGCACCAGGG-3′ | (SEQ ID NO:18) |
| and | |
| b-actin, | |
| 5′-ATCATGTTTGAGACCTTCAA-3′ | (SEQ ID NO:19) |
| and | |
| 5′-CATCTCTTGCTCGAAGTCCA-3′. | (SEQ ID NO:20) |

### Example 2

### Macrophage/Prostate Cancer Cell Interactions Causes Resistance to SARMs

Based on previous studies indicating that a p50 regulated gene, KAI1, can be derepressed in response to pro-inflammatory cytokines (Baek et al 2002) and because activated macrophages are a source of IL-1, we examined whether macrophage/tumor cell interactions might provide a partial explanation for SARM resistance in prostate cancer biology. We examined potential direct interactions between macrophages and prostate cell lines, initially using RWPE1 transformed prostate cells. The results of our experiments are shown in FIGS. 1A-1E.

More specifically, FIG. 1 shows that macrophage-prostate cell interaction converts an AR antagonist to an agonist. FIG. 1A are tissue microarrays (A302) from prostate cancer patients that were stained with macrophage-specific marker CD68. Representative stainings of cancer tissues and patient-matched pathologically benign tissue are shown. CD68+ cells appear as a dark grey color. Higher magnification showed a specific macrophage-cancer cell interaction. Panel B shows quantitative evaluation of infiltrating macrophages in prostate tissues of 32 patients. Values are means ±S.E.M. and P value was calculated by Student's t test. In Panel C, interaction between fluorescence-labeled Raw 264 macrophages and RWPE1 transformed prostate cells upon stimulation is shown. DAPI staining showed the cell nuclei. Panel D depicts quantitative analysis of relative fluorescence that represents the amount of macrophages engaged to prostate cells in Panel C. One representative of three independent experiments is shown. Panel E shows the results when RWPE1 cells were treated with TNFα and/or incubated with Raw 264 macrophages. Then RWPE1 cells were single-cell microinjected with a lacZ reporter driven by the PSA promoter and treated with ligands. Only under the condition (TNFα+macrophage) that permits enhanced macrophage/prostate cell interaction an AR antagonist converted to an agonist.

Using fluorescent-labeled THP-1 macrophages, we found a specific RWPE1/macrophage interaction (FIG. 1A). Pretreatment of RWPE1 cells with TNFα, LPS, or IL-1 induced the expression of VCAM-1 (data not shown) and enhanced the engagement of macrophages (FIG. 1A,B). We found that these interactions were blocked by addition of a specific VCAM-1 monoclonal IgG, demonstrating the importance of VCAM-1 in macrophage/prostate cell interaction (FIG. 1A,B). These data raised the possibility that such interactions might also operate in vivo in prostate cancer. We therefore examined tissue arrays containing patient-matched sections with both normal and cancer-containing regions of prostates, stained with CD68, a specific marker of macrophages (Holness and Simmons, 1993; Martinez-Pomares et al., 1996). This revealed that virtually 100% of the samples exhibited macrophage infiltration, as well as stromal interactions with macrophages (FIG. 1C). There was much less interaction between macrophages and pathologically “normal” cellular areas in the resected tumors (FIG. 1C,D).

Therefore, we considered that macrophage/prostate cancer cell interactions represent a possible in vivo mechanism leading to SARM resistance in prostate cancer. We explored whether the direct cell-to-cell interactions that we observed between macrophages and prostate cancer cells might cause changes in the activity of SARMs based on macrophage activation and release of pro-inflammatory cytokines. Nuclear microinjection studies were performed in RWPE1 cells using a Prostate Specific Antigen (PSA) promoter/reporter construct. In these experiments, the prostate cells were pretreated with TNFα where indicated, under conditions that lead to the expression of VCAM-1 and the binding of subsequently added THP-1 cells. Following microinjection of the PSA construct, THP-1 cells were added to the prostate cells for 6 hours, and then either DHT or Bicalutamide were added for six hours. After washing, the RWPE-1 cells were stained with Xgal to evaluate reporter activity. We observed the expected activity of the PSA reporter whether or not the cells had been pretreated with TNFα (FIG. 1E), with a robust increase in activity after DHT, but not Bicalutamide. Addition of macrophages did not change this response, except in RWPE1 cells that had been pretreated with TNFα. In this case, there was reporter activity in the presence of Bicalutamide, which was reversed by the addition of VCAM-1 antibody prior to macrophage addition.

The studies reported in this invention have provided evidence that interactions between macrophages and specific cancer cells, including prostate cancers, serve to mediate specific aspects of tumor behavior and responses to androgen receptor antagonists based on an evolutionarily-conserved sensor system. Thus, in addition to clear roles of macrophages in atherosclerosis (reviewed in Li and Glass, 2002), presumptive roles in diabetes mellitus, and correlating to effects on vascularization of tumors (reviewed in Coussens and Werb, 2002), macrophages are capable of serving as an important aspect of SARM resistance in prostate cancer. Just as the presence of monocyte/macrophage infiltration in the adipose tissue of diabetic patients proved to be of functional importance, similar functional consequences are likely to exert mechanistic roles in the progression of many types of tumors. Here, we have found that macrophage/prostate cancer interactions appear almost universal in most clinical tumor samples, which could implicate roles of macrophage-produced cytokines as a virtually ubiquitous signal in dictating prostate cancer cell responses. Prostate cancer cells are reported to be capable of producing chemoattractants, e.g. GM-CSF, which might be the initial step for macrophage recruitment (Chung et al., 1999). We have provided evidence that this macrophage/prostate cancer cell interaction, ultimately mediated via VCAM-1-dependent adhesion, caused activation and production of cytokines, including IL-1β, by the macrophages, which are sufficient to cause resistance to antagonists (SARMs). These data suggest that the interaction between these two cell types serves, at least as an important component of the “resistance” events in prostate cancer. Thus, in addition to roles in induction and surveillance, the recruitment and activation of macrophages is a key aspect of many diseases. Because we have found that peptides corresponding to the L/HX₇LL motif of AR or ERα can block macrophage-dependent resistance, this peptide inhibitor serves as a prototype for identifying nonpeptide anti-agonists that might act to prevent inflammatory cytokine-dependent switch in SARM or SERM function and hence “block” resistance. Our data also support the idea that II-1β-mediated MEKK1 activation is likely to be the major macrophage-induced pathway for SARM resistance, reflecting an evolutionarily-conserved mechanism required for effective reproduction.

### Example 3

### IL-1β Converts Androgen Antagonists to Function as Agonists

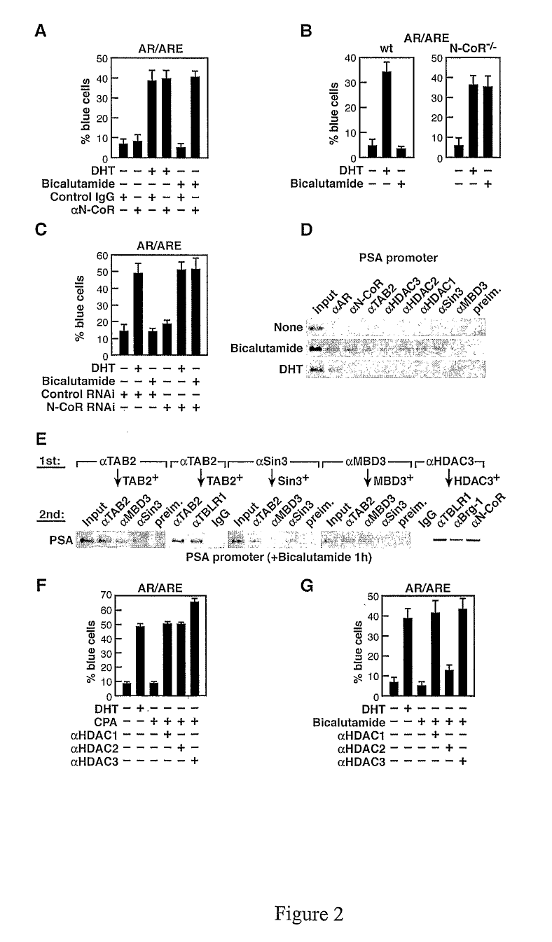

Because cytokines have been shown to exert effects upon the activity of corepressors including N-CoR (Baek et al 2002), we then conducted mechanistic studies designed to investigate the role of N-CoR and other repressors in SARM-dependent repression of the transcriptional activity of androgen receptor. Single cell nuclear microinjection of specific anti-N-CoR IgG abolished the repression of a reporter under control of an androgen receptor response element in the presence of bicalutamide (FIG. 2A). We also found that androgen receptor antagonists behave as agonists in MEFs from N-CoR^{-/-} mice, stimulating target gene expression (FIG. 2B). These results were confirmed using an independent approach, with validated siRNAs against N-CoR (Baek et al., 2002), which consistently reversed bicalutamide-dependent repression function (FIG. 2C). This is consistent with the finding that failure of N-CoR recruitment occurs in aggressive tumors with AR overexpression (Chen et al., 2004), leading to antagonist resistance.

We were able to observe the presence of individual components of many distinct biochemical complexes of N-CoR (Guenther et al., 2000; Li et al., 2000, Zhang et al., 2002, Yoon et al., 2003; Alland et al., 1997; Heinzel et al., 1997; Nagy et al., 1997, Underhill et al., 2000, Humphrey et al., 2001, Jepsen and Rosenfeld, 2002) on the prostate-specific antigen (PSA) promoter (Cleutjens et al., 1996) in a bicalutamide-dependent manner using chromatin immunoprecipitation (ChIP) assay (FIG. 2D), but it was unclear whether these complexes are independently recruited as distinct N-CoR complexes or combinatorially recruited as a holocomplex. Sequential ChIP assay of the PSA promoter revealed that many components that mark the independently-isolated N-CoR complex, including the TAB2/HDAC3-, the TBL1/TBLR1-, the Sin3-, and the Brg1-containing complexes were apparently co-recruited in bicalutamide-dependent repression (FIG. 2E). In contrast, an MBD3-containing complex (Tong et al., 1998; Zhang et al., 1999) may not be simultaneously assembled with other components such as the Sin3 complex on the PSA promoter (FIG. 2E).

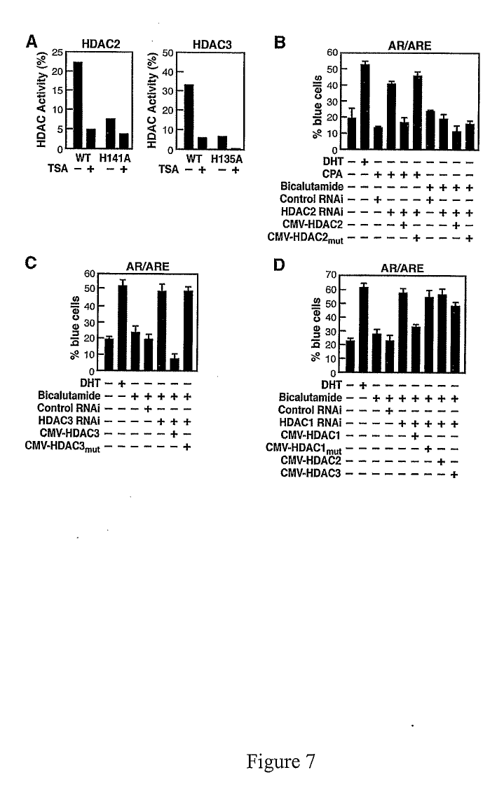

Injection of specific purified IgGs against HDAC1, HDAC2, or HDAC3 showed that blocking the actions of any of these HDACs relieved cyproterone (CPA)-dependent repression, while HDAC1 and HDAC3, but not HDAC2, were required for bicalutamide mediated repression (FIG. 2F,G). Because HDACs that do not have functional HDAC activity failed to rescue the knockdown effects of each HDAC siRNA in the presence of bicalutamide or CPA (FIG. 7A,B,C), and functional HDAC2 and HDAC3 failed to compensate for the HDAC1 activity (FIG. 7D), distinct HDAC enzymatic activities are suggested to be combinatorially required for maintenance of the SARM effects on AR. These results suggest that distinct HDAC-specific substrates might be required for effective SARM-dependent repression.

More specifically, FIG. 2 depicts N-CoR corepressors in androgen receptor antagonist actions. Panel A shows single-cell nuclear microinjection of anti-N-CoR IgG largely relieved the bicalutamide-dependent repression of a lacZ reporter driven by ARE sequences. Panel B shows that in N-CoR−/− MEFs, bicalutamide acted as an agonist. Panel C shows that microinjection of N-CoR siRNA resulted in a switch from repression to activation. Panel D shows a chromatin immunoprecipitation (ChIP) assay of AR occupancy on the PSA promoter in response to hormone treatment. LNCaP cells cultured in the presence or absence of bicalutamide (10 uM) or 100 nM of DHT for 1 hr, and soluble chromatin was prepared after formaldehyde-cross linking and sonication. Specific IgGs against AR, N-CoR, TAB2, HDAC3, HDAC2, HDAC1, Sin3, or MBD3 were used to immunoprecipitate protein-bound DNA fragments. Panel E shows a serial 2-step CHIP assay to determine whether the different co-repressor complexes are assembled on the same promoter. Soluble chromatin was prepared from LNCaP cells treated with bicalutamide for 1 hr, and was first immunoprecipitated with indicated antibodies (1^{st} IP). The bound material was eluted and divided into several aliquots, and re-immunoprecipitated with antibodies as shown (2^{nd} IP). Panel F shows microinjection of IgGs against HDAC1, HDAC2, or HDAC3 fully relieved the repression by CPA-bound AR. Panel G shows that, in response to bicalutamide, HDAC1 and HDAC3, but not HDAC2, were required for repression.

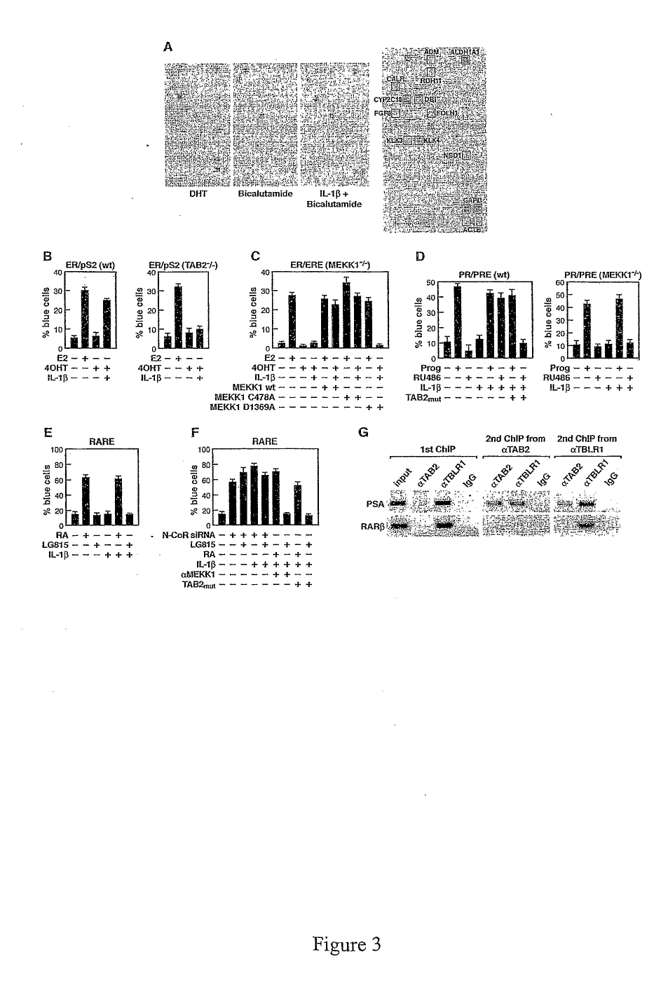

FIG. 3 shows that IL-1β converts AR antagonists to agonists. Panel A shows that pretreatment of cells with IL-1 β abolished CPA or bicalutamide-mediated repression of a reporter containing ARE, and nuclear microinjection of CMV-N-CoR expression plasmid restored the antagonist function. Panel B shows a ChIP assay on the PSA promoter in LNCaP cells in the presence of CPA and IL-1β. LNCaP cells were pretreated with CPA for 1 hr, and dismissal of co-repressor complex was assessed at indicated times after IL-1β treatment. Panel C shows LNCaP cells that were maintained in charcoal-stripped medium, and exposed to CPA in the absence or presence of IL-1β. Cells were stained with antibodies directed against the N-CoR, and the fluorescence-conjugated secondary antibody was visualized using deconvoluting microscopy. Merged images with nuclear staining with DAPI are shown. Panel D shows the results of microinjection of anti-TAB2 and anti-MEKK1 IgGs into Rat-1 cells with AR and a LacZ reporter containing three ARE controlling a minimal p36 promoter, in the presence or absence of DHT or CPA. 5 ng/ml of leptomycin B (LMB), a nuclear export inhibitor, was also used for 30 min followed by treatment with 10 ng/ml of IL-1β. Panel E shows a reporter under the control of ARE was microinjected into either MEKK1−/− or wild-type (wt) MEFs, and the effects of treatment of DHT, CPA, or IL-1β were tested in the presence of AR. Panel F shows that in TAB2−/− MEFs, IL-1β-dependent androgen-like activity of CPA was abolished. Panel G shows the role of TAB2 in bicalutamide-dependent repression of AR function. Using TAB2−/− MEFs, an AR-dependent reporter gene is not activated by bicalutamide/IL-1β co-treatment, unless wt TAB2 is expressed, or N-CoR/SMRT are depleted by 48 h of specific siRNAs. Panel H shows that in MEKK1−/− MEFs, bicalutamide acts as antagonists upon bicalutamide/IL-1β co-treatment unless MEKK1 or a C478A mutation abrogating its ubiquitin ligase function, is added.

FIG. 7 shows combinatorial functions of HDACs in SARM-dependent repression. Panel A shows that HDAC activity was measured following immunoprecipitation in the absence or presence of TSA, HDAC inhibitor, using fluorogenic HDAC substrate as previously described (Hoffmann et al., 1999). Evaluation of the effects of microinjected HDAC2 siRNA (Panel B), HDAC1 siRNA (Pane C) or HDAC3 siRNA (Panel D) for repression by CPA or bicalutamide-bound AR. Expression plasmids for either wild type or enzymatically inactive HDACs were also used in rescue experiments. Injection of HDAC siRNAs caused derepression of SARM-dependent repression, and injection of functional HDAC expression plasmids specifically restored the repression function.

Consistent with the potential actions of pro-inflammatory cytokines secreted in response to macrophage/prostate cancer cell interactions in modulating tumor progression and drug resistance, we found that IL-1β addition to CPA or bicalutamide-bound AR “switched” the AR to function as an agonist, which could be overcome by overexpression of N-CoR (FIG. 3A).

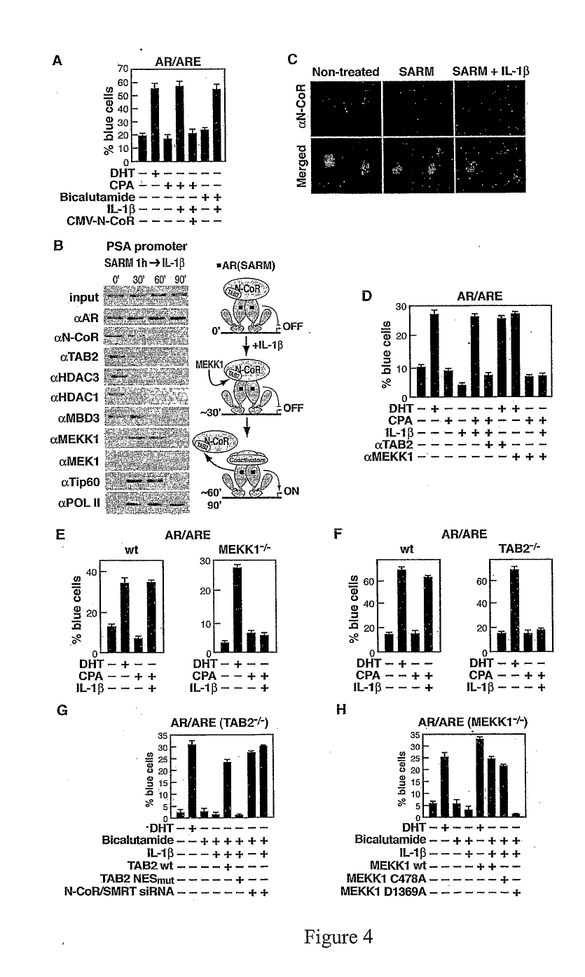

Upon stimulation by IL-1β, there was a progressive and complete dismissal of all components of the N-CoR-containing complex recruited by SARM-bound AR for the PSA promoter assessed using ChIP assay (FIG. 3B). Consistent with the observation that TAB2 is a substrate for MEKK1, potentially causing the exposure of a nuclear export signal (Baek et al., 2002), IL-1β-dependent loss of the N-CoR complex from AR was accompanied by a transient recruitment of MEKK1 (FIG. 3B). In LNCaP prostate cancer cells, N-CoR was detected in both nuclear and cytoplasmic locations in either non-treated or SARM-treated LNCaP cells, but N-CoR became more selectively localized to cytoplasm after IL-1β treatment (FIG. 3C). Injection of either αTAB2 or αMEKK1 IgG abolished the activation of an androgen-dependent reporter in the presence of IL-1β and SARM (FIG. 3D). Similarly, IL-1β-dependent agonistic activity of SARMs was abolished using MEFs either from MEKK1 gene-deleted mice (FIG. 3E) or from TAB2 gene-deleted mice (FIG. 3F), supporting the model that MEKK1-dependent recruitment to TAB2 is required for the removal of N-CoR complexes, resulting in relief of repression by SARMs on AR target gene promoters. In TAB2−/− MEFs, the SARM bicalutamide acted as an antagonist even with IL-1β treatment unless TAB2 was reexpressed or N-CoR/SMRT expression was abrogated by microinjection of both specific siRNAs (FIG. 3G) (Perissi et al., 2004). In MEKK1−/− MEFs, we rescued SARM-bound AR activation by IL-1β by expression of wild-type MEKK1 or an MEKK1 protein with a point mutation to block its internal ubiquitin ligase activity (C478A) (Lu et al., 2002). MEKK1 harboring a mutation blocking its protein kinase activity (D1369A) failed to function in AR derepression (FIG. 3H). To investigate whether effects of IL-1β on SARM actions represented a general effect, or were gene-specific, we performed an RNA profiling experiment, finding that virtually all DHT-stimulated gene transcripts recorded were also induced by bicalutamide in the presence of IL-1β, suggesting that this strategy regulates most or all AR-dependent genes (FIG. 4A).

FIG. 4 shows steroid hormone receptor-specific derepression by IL-11. Panel A shows expression profiling of AR target genes in LNCaP cells treated with DHT, bicalutamide and bicalutamide/IL-1β. Upregulated genes upon normalization were marked. Panel B shows the effects of IL-1β on 4-OHT-mediated pS2 promoter-dependent reporter in wt or TAB2−/− MEFs. Panel B shows the ability of MEKK1 expression vectors to rescue 4-OHT-mediated activation in response to IL-1β. Wild-type MEKK1, C478A MEKK1 (mutant of ubiquitin ligase activity), or D1369A MEKK1 (mutant of kinase activity) was used to rescue IL-1β-dependent activation of the pS2 promoter by 4-OHT in MEFs from MEKK−/− mice. Panel C shows the effects of RU486 on progesterone receptor-dependent activation of a PRE-dependent reporter in response to IL-1β, using single cell nuclear microinjection assays. In MEKK1−/− MEFs, IL-1β did not stimulate activation in response to IL-1β. Panel D shows that the absence of RAR agonist, or the RAR antagonist LG815, fails to confer activation by IL-1β in Rat1 cells. Panel E shows that antibody against MEKK1 and a dominant negative TAB2 (NESmut) have no effect on LG815 or RA actions in response to IL-1β. Panel F shows a two-step ChIP analysis of RARβ and PSA promoters in LNCaP cells, showing that PSA promoter converts TBLR1 and TAB2 when cells are treated with CPA (1 uM) for 1 hour, while RARβ promoter only recruits TBLR1, but not TAB2 in cells cultured in charcoal-stripped medium.

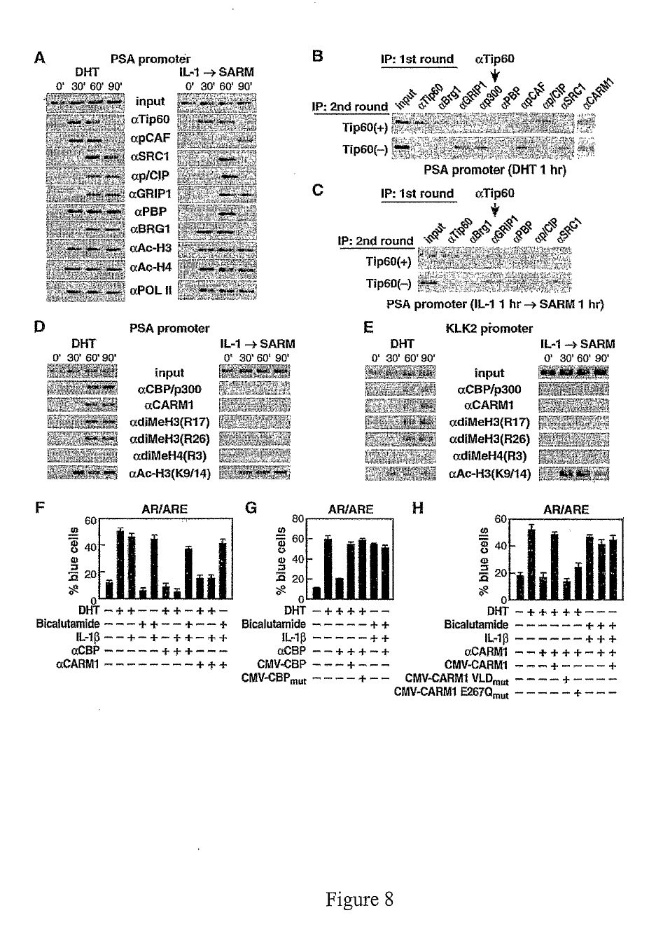

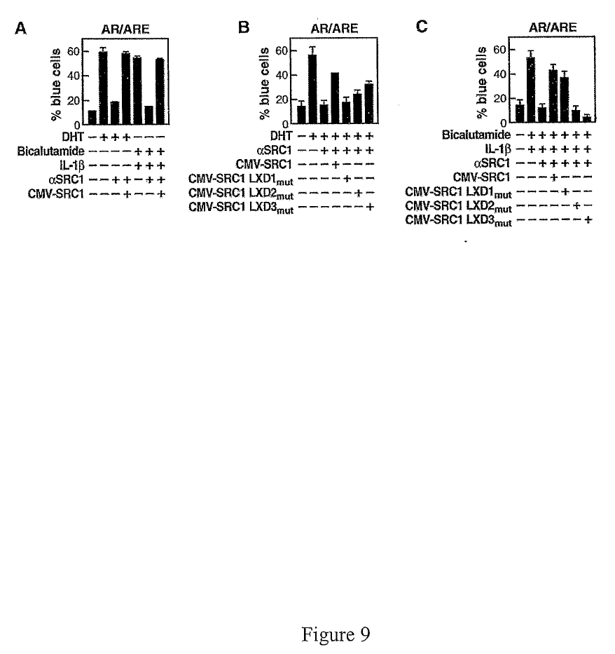

Upon loss of corepressors, SARMs functioned as agonists and generally exhibit the same sequential coactivator complex recruitment pattern elicited by DHT-bound AR on the PSA and KLK2 promoters (FIG. 8A,B,C), analogous to sequential coactivator recruitment events on the pS2 promoter in response to estrogen (Metivier et al., 2003). Interestingly, however, SARMs in the presence of IL-1β, failed to recruit either CBP/p300 or CARM1 and did not exhibit methylation of histone H3 at R17 and R26, although maintaining acetylation of histone H3 at K9 and K14 (FIG. 8D,E). Indeed, microinjection of specific αBP/p300 IgGs or αCARM1 IgGs or siRNAs markedly inhibited DHT-dependent activation, but exerted no effect on the activation by IL-1β and SARMs (FIG. 8F, G, H, and data not shown). While the p160 coactivator SRC-1, which recruits both CBP/p300 and CARM1 (Chen et al., 1999), is required for both agonist and SARM/IL-1β-dependent activation (FIG. 9A), different LXXLL motifs (Le Douarin et al., 1995; Heery et al., 1997; Torchia et al., 1997; Ding et al., 1998; Darimont et al., 1998; Nolte et al., 1998; Shiau et al., 1998; McInerney et al., 1998; Voegel et al. 1998; McInerney et al., 1998) were required for the DHT versus SARM/IL-1β-mediated activation (FIG. 9B,C).

FIG. 8 shows evaluation of the roles of coactivators. Panel A shows a ChIP assay to monitor the occupancy of PSA promoter by Tip60, pCAF, SRC1, p/CIP, GRIP1, PBP, BRG1, acetylated histones H3/H4, and RNA polymerase II (pol II) at the indicated times after treatment with either DHT or IL-1β and SARM. Soluble chromatin was prepared from LNCaP cells treated with either DHT for 1 hr (Panel B), or IL-1β and CPA for 1 hr (Panel C). An aliquot was first immunoprecipitated with IgG against Tip60 (1^{st} IP). The bound materials and supernatant were collected and re-immunoprecipitated with IgGs against Tip60, Brg1, GRIP1, p300, PBP, pCAF, pCIP, SRC1, or CARM1. Panel D shows ChIP analysis of CBP/p300, CARM1 and other histone modifying factors on PSA promoter following treatment of either DHT or IL-1β and SARM. Panel E shows that KLK2 promoter, another AR responsive promoter, was examined after challenging with SARM and IL-1β revealing that CBP/p300 were not recruited on KLK2 promoter. Panel G shows that injection of anti-CBP IgG or anti-CARM1 IgG blocked the DHT-dependent activation of a reporter containing ARE, but not the IL-1β/SARM-dependent activation. After microinjection of anti-CBP IgG, plasmid rescue experiments were performed as indicated. Panel H shows that the function of CARM1 was assessed in cells injected with the ARE reporter. Rescue experiments with CARM1 expression plasmids confirm a requirement for the functional methyltransferase activity.

FIG. 9 shows the role of the LXXLL motif of SRC-1 in agonist-mediated activation and IL-1β-induced derepression of AR. Panel A shows injection of anti-SRC-1 IgG blocked both the DHT-dependent and bicalutamide/IL-1β-dependent activation of a reporter containing ARE. Panels B and C show that after microinjection of anti-SRC-1 IgG, each LXXLL mutant of SRC-1 plasmid rescued the activation in the presence of DHT or bicalutamide and IL-1β.

### Example 4

### Molecular Mechanisms of Steroid Hormone Receptor-Specific Derepression by IL-1β

Analogous to events on SARM-bound AR, IL-1β-dependent conversion of SERMs (4-OH tamoxifen) to activators of estrogen receptor-α (ERα) also failed to occur in cells null for either MEKK1 or TAB2 (FIG. 4B,C). Similarly, antagonists of progesterone receptor (PR) were also switched to agonists in response to IL-1β and both MEKK1 and TAB2 were required for agonistic actions of progesterone antagonists (FIG. 4D). In contrast, even in the same LNCaP cells, RARα-dependent repression was not altered by IL-1β either in the absence of ligands or even with binding of the RARα antagonist LG815 (FIG. 4E). Moreover, RARα-dependent activation or repression was not altered by disrupting TAB2 or MEKK1 functions using siRNAs or dominant negative mutant counterparts (FIG. 4F). ChIP assay revealed that the N-CoR holocorepressor complex, including TBLR1, mSin3A/B-containing complexes and HDAC3, were present on the RARβ, promoter (FIG. 4G). On this RARα target, TAB2 and IL-1β-dependent recruitment of MEKK1 could not be detected, either in the presence or in the absence of antagonists. This is in contrast to the presence of TAB2 in the N-CoR holorepressor complex on the PSA promoter in response to SARMs (FIG. 4G and data not shown).

The receptor specificity of the IL-1β response is consistent with the hypothesis that IL-1β-dependent dismissal of N-CoR holocorepressor complexes on androgen, estrogen, and progesterone receptors requires the presence of a component of the N-CoR complex, TAB2, that is not successfully recruited to unliganded or to antagonist-bound RXR/RAR complexes, raising the question why TAB2 is recruited to some nuclear receptors, but not to others. One striking difference between sex hormone receptors and retinoic acid and thyroid hormone receptors is the relative importance of the N-terminus in gene activation events, based on interactions between N- and C-terminus (Kraus et al., 1995; Langley et al., 1995; Kemppainen et al., 1999; Tetel et al., 1999) and the presence of distinct activation domains (AF1) in the N-terminus of estrogen and androgen receptors (Alen et al., 1999; Bevan et al., 1999; Tremblay et al., 1999; Kobayashi et al., 2000; Metivier et al., 2001). Indeed, the N-terminal domain of ERα is 185 aa (Gustafsson, 1999) and that of AR is 537 aa (Quigley et al., 1995), compared to a relatively short (87 aa) RARα N-terminus (Leid et al., 1992).

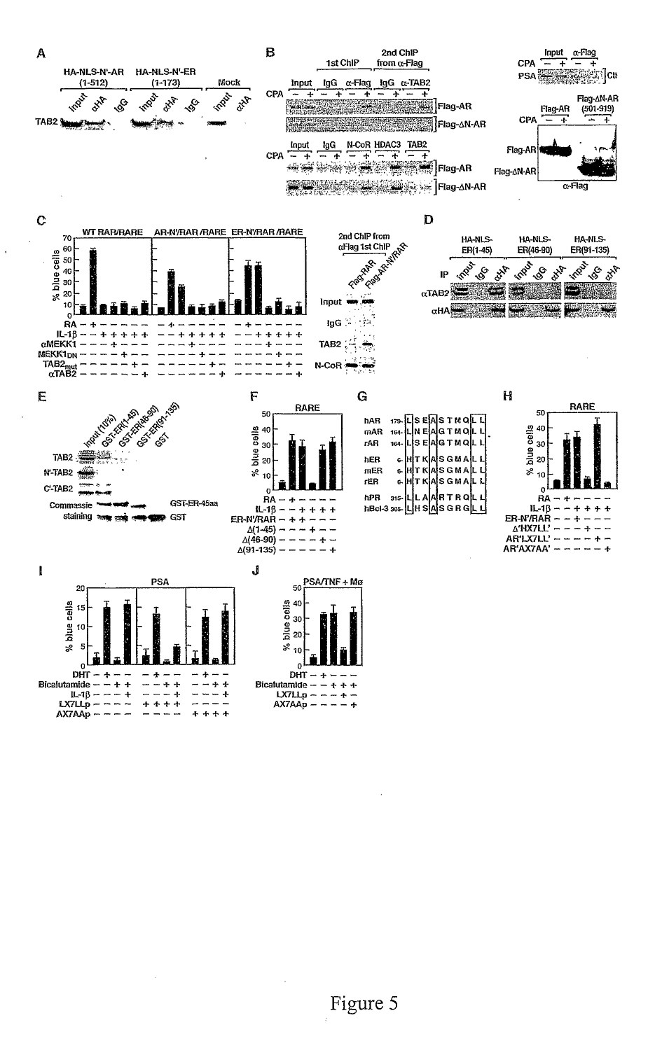

We therefore examined the possibility that the N-termini of androgen, estrogen, or progesterone receptors harbored the key for inclusion of TAB2 into the N-CoR holocorepressor complex. Consistent with this model, we found that TAB2 can interact with the N-terminus of either the androgen (aa 1-512) or estrogen receptor (aa 1-173) in coimmunoprecipitation assays (FIG. 5A). Further, with removal of the androgen receptor terminus (aa 2-500), in comparison to androgen holoreceptor, there was no change in the recruitment of receptors themselves and other components of the N-CoR holocomplex, including HDAC3, TBLR1, and mSin3A/B in response to SARMs, but there was a loss of ability to recruit TAB2 (FIG. 5B, and data not shown). A similar result was found with the estrogen receptor, where the presence of the N-terminus (aa 1-170) is required for recruitment of TAB2, but not components of the N-CoR holocomplex, in response to SERMs (data not shown). Finally, with replacement of (or addition to) the RARα N-terminus (aa 1-60) with that of either the androgen receptor (aa 1-512), or estrogen receptor (aa 1-173), the RARα fusion proteins (AR-N′/RAR, ER-N′/RAR) can now exhibit MEKK1- and TAB2-dependent activation with RARα antagonist or in the absence of ligands in response to IL-1β (FIG. 5C and data not shown). Further, a serial two-step ChIP assay confirmed that N-CoR and TBLR1 components were present in both RARα and the AR-N′/RARα fusion receptor, and that TAB2 could be recruited only to the fusion receptor (FIG. 5C and data not shown), confirming that the steroid hormone receptor N-termini are required for TAB2 recruitment to N-CoR complex during repression. Retinoic acid stimulated the chimeric RARα, but neither E₂ nor DHT exerted activation effects (FIG. 5C and data not shown).

To localize a potential interaction domain(s), HA-tagged fragments of ERα N-terminal sequences, fused to an NLS, were transfected into 293 cells and Western Blot analysis was performed on αA immunoprecipitated material from nuclear extracts using αTAB2 IgG. These studies revealed ERα N-terminus aa 1-45 was sufficient to permit TAB2 binding (FIG. 5D). Bacterially-expressed Erα fragments were used to evaluate if this might reflect a direct interaction; again the initial 45 aa of ERα N-terminus was sufficient to cause direct interactions with TAB2. Further mapping revealed that this interaction was dependent upon the C-terminus (aa 400-693) of TAB2, a region that also harbors the critical regulatory MEKK1 phosphorylation site (aa 419-423) and the NES site (aa 547-561) (FIG. 5E). Finally, deletion of aa 1-45 of the ERα N-terminal sequence within the ERα-N′/RARα chimeric receptor caused loss of IL-1β response while deletion of aa 46-40 or aa 91-135 failed to block IL-1β induced activation (FIG. 5F).

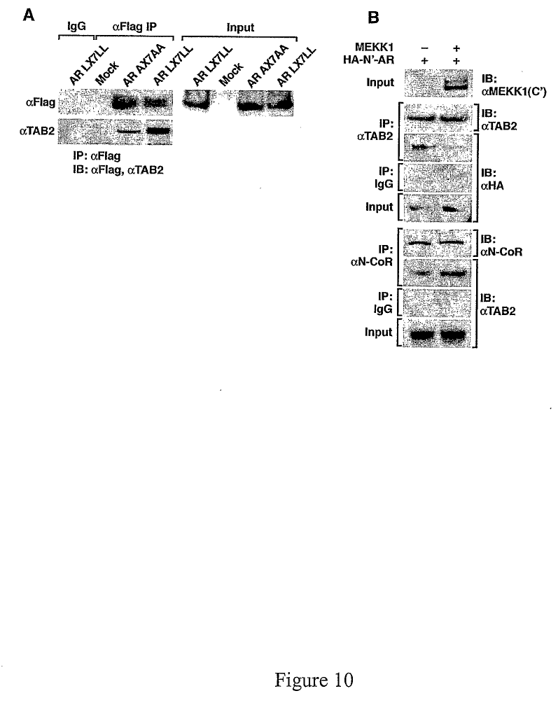

Within the TAB2 regulatory N-terminal sequences of AR and of ERα, we identified a sequence, L/HXXAXXXXLL (SEQ ID NO:21) (referred to as L/HX₇LL (SEQ ID NO:22)), conserved between species and between estrogen, androgen and progesterone receptors (FIG. 5G), and also related to a sequence present in Bcl-3, which is recruited to the p50 site of KAI1, a site that also recruits TAB2 with the N-CoR holocomplex and which is derepressed in response to IL-1β. These residues are predicted to form a specific putative helical structure, based on biochemical studies on AR N-terminus (Reid et al., 2002). To further test the hypothesis that this site is required or even sufficient to confer interactions with TAB2 and exert the IL-1β action, we deleted the N-terminal 15 aa including HX7LL (SEQ ID NO:23) sequence from the ERα-N′/RARα fusion protein (DHX7LL (SEQ ID NO:24)), finding that deletion of the sequence causes loss of its ability to transfer activation of RARα by IL-1β (FIG. 5H). Replacement of the HX7LL (SEQ ID NO:23) sequence of ERα with the corresponding LX7LL (SEQ ID NO:25) sequence of AR, but not an AX7AA (SEQ ID NO:26) mutant AR sequence, is sufficient to restore IL-1β-mediated activation of ERα-N′/RARα on an RARE or on the RARβ promoter (FIG. 5H and data not shown). As an independent confirmation, we transfected 293 cells with the Flag-tagged AR wild-type (LX7LL (SEQ ID NO:25)) or mutant (AX7AA (SEQ ID NO:26)) motif-containing RARα fusion receptors used in the reporter assays and performed a co-immunoprecipitation analysis, finding a decreased interaction of the mutant fusion receptor with TAB2 (FIG. 10A). Therefore, the ability to include TAB2 into the nuclear receptor-recruited N-CoR holocorepressor complex is conferred by a specific, evolutionarily-conserved sequence that is present in both AR and ERα, and which underlies the ability of gene targets of these receptors be induced by a pro-inflammatory cytokine.

FIG. 10 shows roles of N-terminal L/HX₇LL (SEQ ID NO:22) motif of steroid hormone receptors and MEKK1 in the interactions of TAB2/nuclear receptors and TAB2/N-CoR. Panel A shows co-immunoprecipitation of endogenous TAB2 and Flag-tagged ERα-N′(16-174)/RARα(61-462) fusion receptor containing AR wt (LX7LL (SEQ ID NO:25)) or L to A mutant (AX7AA (SEQ ID NO:26)) motif. The plasmids are the same ones used in the single cell nuclear microinjection experiments shown in FIG. 5H. Panel B shows MEKK1 and HA-NLS-N′-AR were overexpressed in 293 cells and nuclear extracts were prepared for Western blots and immunoprecipitations. The N-terminus-truncated MEKK1 (M.W. ˜98 kDa) was detected by an antibody specifically recognizing MEKK1 C-terminus. Co-immunuoprecipitation assays were carried out by immunoprecipitating endogenous TAB2 and N-CoR and then immunoblotting TAB2/HA-NLS-N′-AR and N-CoR/TAB2, respectively, revealing that activation of MEKK1 leads to decreased association between TAB2 and AR N-terminus, and increased interaction between TAB2 and N-CoR.

To test whether the L/HX₇LL (SEQ ID NO:22) motif might actually be sufficient to exert IL-1β activation of androgen target genes by SARMs, we used the single cell nuclear microinjection assay to introduce a 14mer synthetic peptide encompassing either the AR LX7LL (SEQ ID NO:25) motif or the same sequence with L to A substitutions (AX7AA (SEQ ID NO:26)) into cells expressing PSA promoter- or ARE-dependent reporters. We found that the AR N-terminal LX7LL (SEQ ID NO:24) peptide specifically blocked IL-1β-mediated activator function of bicalutamide, while the mutated AX7AA (SEQ ID NO:26) sequence failed to inhibit induction (FIG. 5I). Therefore, this sequence is specific and sufficient to mediate TAB2 recruitment and IL-1β-mediated derepression, respectively. Finally, to test whether the L/HX₇LL (SEQ ID NO:22) motif is also important for macrophage-induced derepression in prostate cells, we performed nuclear microinjection of the AR-N′ LX7LL (SEQ ID NO:25) 14-mer peptide into RWPE1 cells, pre-treated with TNFα to induce VCAM-1 and interaction with THP-1 macrophages as described in FIG. 1. We then assessed the effects of DHT or bicalutamide treatment on PSA promoter-dependent reporter induction. Intriguingly, the LX7LL (SEQ ID NO:25)-containing peptide, but not the AX7AA (SEQ ID NO:26) mutant, blocked the macrophage-induced agonistic “switch” of bicalutamide (FIG. 5J), implying a potential therapeutic approach to treating hormone resistance induced by macrophage/cancer cell interaction in prostate cancer.

FIG. 5 shows the evolutionarily conserved N-terminal L/HX₇LL (SEQ ID NO:22) motif of steroid hormone receptors is important for TAB2 recruitment and IL-1β-induced derepression. Panel A shows immunoprecipitation of extracts of 293 cells transfected (or mock transfected cells as the control) with N-terminus of AR (1-512 aa) or ERα (1-173 aa) (expressed as HA-tagged, NLS-containing fusion proteins) by α-HA antibody. The specific TAB2 interaction with AR or ERα N-terminus was detected by Western blot. Panel B shows the role of AR N-terminus in TAB2 recruitment to SARM-bound AR in target genes (PSA). Expression vectors encoding Flag-tagged full-length AR or DN-AR (501-919) were transfected into LNCaP cells, and cells were treated with CPA (1 uM) for 1 h if needed. Then a 2-step ChIP was performed, first using αFlag IgG, and then αTAB2 IgG. Lower panels: CPA-treated or untreated LNCaP cells transfected with AR or DN-AR Flag-tagged vectors were examined by a 2-step ChIP (first αFlag and then α-corepressors) on the PSA promoter for binding of N-CoR, HDAC3, and TAB2. Mock-transfected cells were used as the control for ChIP analysis. Western blot confirmed equivalent expression of AR and AN-AR. Panel C shows single cell nuclear microinjection assays on fusion proteins of N-terminus of AR (aa 1-512) or ERα (aa 1-173) and ΔN-RARα (61-462), showing an IL-1β-induced, MEKK1- and TAB2-dependent activation of an RARα-dependent reporter in the absence of ligand. A 2-step ChIP showed a specific co-occupancy of TAB2 with AR-N′/RARα fusion receptor but not the wild-type RARα. Panel D shows co-immunoprecipitation of endogenous TAB2 with HA-tagged different fragments of ERα N-terminus. Panel E shows GST pull-down assays using different fragments of ERα N-terminus fused to GST and in vitro translated full-length, N-terminal (aa 1-399) or C-terminal (aa 400-693) TAB2. The Coomassie staining of the purified GST fusion proteins showed a similar amount used in the assays. Panel F shows that deletion of the 1-45 aa of Erα N-terminus, but not other fragments, lost the IL-1β-mediated derepression of the ERα-N′/RARα fusion receptor in the single cell microinjection assays using an RARα-dependent reporter. Panel G shows alignment of L/HX₇LL (SEQ ID NO:22) motif in AR, ERα, PR and Bcl-3 of different species. Panel H shows specific deletion and replacement of the L/HX₇LL motifs or mutant (AX7AA (SEQ ID NO:26)) of Erα and AR in the ERα-N′/RARα fusion receptor in the single cell microinjection assays using an RARα-dependent reporter. Panel I shows that in single cell microinjection assays using PSA promoter-controlled reporter in RWPE1 prostate cells, synthetic 14mer peptide harboring wt LX7LL (SEQ ID NO:25) motif but not the mutant AX7AA (SEQ ID NO:26) abolished the IL-1β-mediated conversion of antagonist to agonist. Panel J shows that, when macrophage/prostate cell interaction was set up as described in FIG. 1, by single cell nuclear microinjection, the 14-mer wt LX7LL (SEQ ID NO:25) and mutant AX7AA (SEQ ID NO:26) peptides were co-injected into RWPE1 prostate cells with a reporter driven by the PSA promoter, and then ligands were added into the medium.

We finally explored the mechanisms by which MEKK1 induces TAB2/N-CoR dismissal from the target gene promoters in response to pro-inflammatory signals, via the suggested MEK1-dependent phosphorylation of TAB2 (Baek et al., 2002). We performed co-immunoprecipitation of endogenous TAB 2, N-CoR and HA-tagged AR N-terminus (1-512) after overexpression of an MEKK1 mutant that results in accumulation of N-terminally-truncated kinase-active form of MEK1 in nucleus (Gibson et al., 1999). We observed that activation of MEKK1 kinase activity inhibits TAB2 interactions with the AR N-terminus, while enhancing its interactions with N-CoR, consistent with its function in removal of the N-CoR holocorepressor complex in derepression (FIG. 10B).

The basic effect of the macrophage/prostate cancer cell interaction is to activate a specific molecular mechanism that dictates a coordinated program of transcriptional response to an inflammatory cytokine elaborated as a result of derepression of a subset of N-CoR/SMRT-repressed genes. This is based on the presence or absence of a specific molecular beacon, TAB2, that acts as a sensor of specific inflammatory signaling pathways. We find that recruitment of an N-CoR “holocorepressor” complex, containing components of many biochemically-defined N-CoR complexes, including multiple HDAC enzymatic activities that are combinatorially required for maintenance of repression, serves to maintain repression on SARM or SERM-bound androgen or estrogen receptors. However, in addition to TBL1/TBLR1-, mSin3-, and Brg1-containing components that are simultaneously recruited with N-CoR to these receptors, it is the inclusion of a specific component, TAB2 that underlies macrophage/prostate cancer cell derepression events. This complexity of the holocorepressor machinery may serve primarily to permit an “integration” of transcriptional responses in response to additional regulatory signals, exemplified by the selective derepression of specific cohorts of N-CoR-repressed genes in response to pro-inflammatory signal-mediated signaling events.

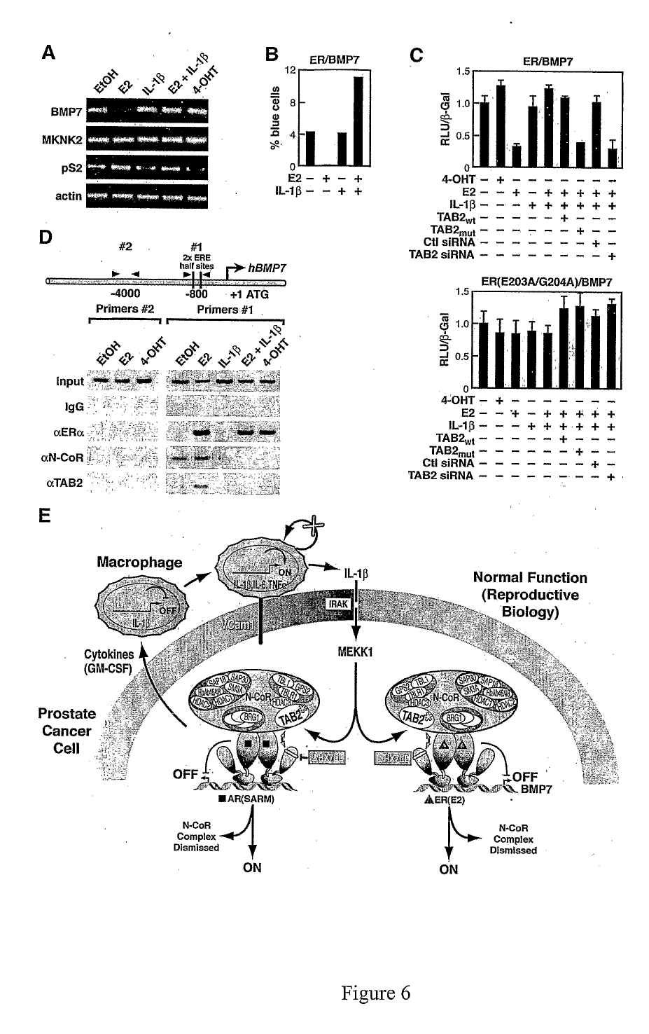

While nuclear receptors capable of recruiting a TAB2 component to the N-CoR complex, as is the case for the androgen, estrogen and progesterone receptors, exhibit IL-1β-dependent derepression, other nuclear receptors, such as retinoic acid receptor, which recruit almost the identical N-CoR holocorepressor complex, with the exception of the TAB2 component, which remains insensitive to the proinflammatory signal. Hence, these receptors are resistant to any derepression actions of IL-1β, whether the receptor is unliganded or bound by an antagonist. Thus, receptor-selective differences in inclusion or exclusion of a specific component of the N-CoR corepressor complex, TAB2, appears to predetermine the patterns of transcriptional response to a specific signal, in this case IL-1β, dictating a precise program of derepression at the level of individual transcription units. All of the derepressed target genes require the presence of TAB2 as a component of the corepressor complex for implementing the IL-1β-dependent pathway of derepression, based on the ability of TAB2 to selectively recruit activated MEKK1, with phosphorylation of a specific residue of TAB2 permitting removal and export of the N-CoR complex. Therefore, we propose that TAB2 serves as a molecular beacon for the cohort of genes that exhibit MEKK1-dependent derepression responses to pro-inflammatory signals, based on the ability to respond to IL-1β, serving to mediate an integrated transcriptional response, linking the ability of specific nuclear receptors and other DNA binding factors such as p50 homodimer targets (Baek et al., 2002) to recruitment of TAB2 into the N-CoR holocorepressor complex (FIG. 6F).

FIG. 6 shows the physiological roles of inflammatory signal dependent derepression in sex steroid receptor regulation. Panel A shows semi-quantitative RT-PCR analysis of Erα target genes upon different treatments. Actin was used as the loading control. Panel B shows single cell nuclear microinjection assays in Rat-1 cells. A BMP7 promoter-controlled LacZ reporter and an ERα expression plasmid were injected into cells. Panel C shows reporter assays using luciferase reporter driven by a human BMP7 promoter sequence (−250 to −1091, numbers indicate relative positions to ATG). Wild-type ERα and Erα (E203A/G204A) were cotransfected with the reporter in 293 cells. The expression levels of wild-type and mutant ERα were similar and the siRNA knock-down of TAB2 was efficient (>70%) (data not shown). Panel D shows ChIP analysis of the BMP7 promoter. Primer pair #1 flanks two putative ERE half-sites, and primer pair #2 (control) flanks a region ˜3 kb upstream of #1. Panel E shows a model of nuclear receptor specific derepression program in response to pro-inflammatory signals. The N-CoR corepressor apparatus appears to be assembled as a holocorepressor complex, however, the N-terminal L/HX₇LL (SEQ ID NO:22) motif of hormone receptors is required for the recruitment of TAB2 into this holocorepressor complex. In turn, the presence of TAB2 acts as a molecular beacon to permit IL-1β-dependent derepression, hence providing an integration of nuclear response to pro-inflammatory signals.

Intriguingly, the molecular basis for recruitment of TAB2 to the N-CoR holocorepressor complex by androgen, estrogen, and progesterone receptors proves to lie in the specific interaction between TAB2 and an evolutionarily-conserved “L/HX₇LL” (SEQ ID NO:22) sequence that permits recruitment of TAB2 to the N-CoR holocorepressor complex in a receptor TAB2/N-CoR ternary complex, present in the N-terminus of these receptors (FIG. 5G), but not in other receptors. The interactions between androgen and estrogen receptor N- and C-terminal domains (Kraus et al., 1995; Langley et al., 1995; Kemppainen et al., 1999), and the role of AF1 in SERM-dependent gene activation events (Berry et al., 1990; Webb et al., 2003), further suggests that these domains impact on critical interactions in gene activation events. Indeed, the N-terminal L/HX₇LL (SEQ ID NO:22) helix may itself have direct molecular interactions with the C-terminal hydrophobic pocket to which N-CoR binds, comprising recruitment of the N-CoR complex by unliganded sex steroid receptors.

While RARα cannot recruit TAB2, and is insensitive to IL-1β signaling, a RARα fusion protein engineered to harbor the estrogen or androgen receptor N-terminus, or a fragment containing the L/HX₇LL (SEQ ID NO:22) TAB2 recognition motif can now recruit TAB2 and exhibit IL-1-sensitive derepression. In the case of p50 gene targets, a similar L/HX₇LL (SEQ ID NO:22) sequence in Bcl-3 appears to mediate recruitment of TAB2. Therefore, the genomic response to IL-1β-dependent actions in derepression appears to be determined by a specific motif that serves to alter the composition of the N-CoR corepressor complex by recruiting the TAB2 component. These rules appear to apply to most, if not all, androgen and estrogen receptor gene targets. Thus, pro-inflammatory cytokines, such as IL-1β, can cause dissociation of the N-CoR holocomplex provided that the TAB2 sensor component of this pathway is incorporated into the recruited corepressor machinery. It is interesting to consider that a molecular explanation of why unliganded sex steroid receptors bind N-CoR so poorly might be that the LX7LL (SEQ ID NO:25) helix in their N-terminus is highly related to the recognition helix in N-CoR responsible for binding to unliganded nuclear receptor C-terminal domain.

While the cytoplasmic function of TAB2 in the IL-1β-induced activation of NF-kB is not impaired in TAB2-deficient embryonic fibroblasts (Sanjo et al., 2003), the nuclear function attributed to TAB2 in permitting IL-1β-dependent corepressor dissociation is lost in the absence of TAB2, based on phosphorylation of TAB2 by MEKK1 (Baek et al., 2002). The MEKK-1-dependent conformational change upon phosphorylation of TAB2 simultaneously causes its more effective disassociation with receptor N-terminus promoting its release from the AR/ER N-terminus, while enhancing the association with the N-CoR complex, which probably enables its role for dismissal of the entire N-CoR “holocorepressor” complex. Thus, DNA binding factor-specific recruitment of specific components of the corepressor holocomplex can serve as the “molecular beacon” for integrating nuclear transcriptional responses of specific signaling pathways, accounting for the resistance of other nuclear receptors to IL-1β signals.

### Example 5

### Physiological Roles of TAB2 and the L/HX₇LL Motif in Sex Steroid Receptor Regulation

While these observations have produced initial evidence for an unexpected mechanism underlying some aspects of SARM/SERM resistance in prostate and breast cancers, and can suggest new possible therapeutic approaches, they simultaneously raise a perplexing issue. Given the evolutionary conservation of the L/HX₇LL (SEQ ID NO:22) motif in specific nuclear receptors, as well as the MEKK1/TAB2 effectors of the pro-inflammatory pathways, it is reasonable to conclude that there must be a physiologically important and currently unappreciated function that underlies this conserved response system, apart from the specific roles in modulating derepression of SARM/SERM actions. While many possible scenarios can be imagined, initial data led us to investigate the putative negative regulation of specific gene expression by 17-β-estradiol. Indeed, a number of genes are reported, directly or indirectly, to be down-regulated by estrogen (Frasor et al., 2003; Lin et al., 2004). One such gene that is negatively-regulated in response to E₂ is the BMP7 gene, and this would be predicated to exert key biological roles in several tissues. We noted that the BMP7 promoter only harbored putative half-ER binding sites, even though E2-bound estrogen receptors can be suggested to inhibit BMP7 expression by direct mechanisms (Kusumegi et al., 2004; Lin et al., 2004).

We used MCF-7 breast cancer cells to test whether ERα could bind to this regulatory gene promoter and therefore caused direct inhibition of BMP7 expression, and whether inflammatory cytokines/macrophages could actually reverse this E₂-dependent gene repression. As shown in FIG. 6A, 17-β-estradiol caused decreased levels of BMP7 transcripts and addition of IL-1β blocked this inhibition by E₂. Single cell nuclear microinjection assays demonstrated that E2 reduced the expression of an hBMP-7 promoter-controlled reporter, while addition of IL-1β actively increased expression over the baseline (FIG. 6B). Using a reporter driven by the hBMP7 proximal promoter we confirmed that in the presence of ERα β-estradiol, but not 4-OHT, decreased the reporter activity, whereas combination of E₂ and IL-1β restored the reporter activity to the baseline (FIG. 6C). A dominant-negative mutant of TAB2, or TAB2 siRNA abrogated derepression of the BMP-7 promoter-dependent reporter, suggesting an important role of TAB2 and the TAB2-interacting L/HX₇LL (SEQ ID NO:22) motif within Erα in this regulation. In contrast, an ERα construct harboring point mutations (E203A/G204A), which prevents DNA binding-dependent activities but not trans-activities (Jakacka et al., 2001), fully abrogated the E2-mediated repression of the reporter, suggesting the DNA-binding ability of ERα is important for regulation of BMP7 expression (FIG. 6C). Consistent with these observations, ChIP analysis revealed the presence of ERα, N-CoR and TAB2 on the BMP7 gene regulatory region in response to E₂. Addition of IL-1β caused dismissal of the N-CoR/TAB2 corepressor complex recruited to BMP7 in the presence of E₂ (FIG. 6D). This is particularly relevant in light of the fact that a key aspect of reproductive regulation, blastocyst implantation, involves a local induction of BMP7 in response to inflammatory signals (Monroe et al., 2000; Paria et al., 2001). This pathway of course is likely to serve other biological functions, perhaps as a release of inhibition by unliganded receptor under specific protein kinase regulation, and exerting roles in the immune system and reproductive tract.

One of the most intriguing challenges raised by these findings has been to uncover the physiological basis for evolutionary conservation of this regulatory mechanism selective for sex steroid receptors. While not being limited to any theory regarding the mechanism of action or relevance of the invention to evolutionary contexts, based on the data presented here, we are tempted to suggest that this mechanism arose in the context of gene inhibition by estrogen, androgen and/or progesterone agonists. Indeed, a number of gene targets have been reported to be negatively regulated by agonists (Frasor et al., 2003; Lin et al., 2004). For example, estrogen appears to cause repression of a number of transcription units, perhaps analogous to events with negative glucocorticoid and thyroid hormone regulation (Chin et al., 1993; Dostert and Heinzel, 2004). Here, exemplified by BMP7, 17β-estradiol causes recruitment of the N-CoR/TAB2 complex and IL-1β reverses this E₂-dependent repression along the TAB2/MEKK1-dependent pathway. This has provided an initial physiological explanation for this evolutionarily-conserved L/HX₇LL (SEQ ID NO:22)-dependent TAB2 recruitment, suggesting that there is derepression of E₂-repressed gene programs in response to inflammatory signals, as well as activation of p50-regulated transcription units may play critical roles in several biological processes. The data are consistent with genetic evidence of interactions between inflammatory response and tumor biology (Greten et al., 2004).

Macrophages are intimately connected with the development of hormone responsive tissues and reproductive organs (Cohen et al., 1999, Gouon-Evans et al., 2000). Here we mechanistically link interactions between macrophages and specific cells to prostate cancer biology and normal reproductive biology. It is intriguing to note that blastocyst implantation into the uterus constitutes an “inflammatory event”, with regulated production of cytokines such as IL-1β and the closely related IL-18 (de los Santos et al 1996, Ostojic et al 2003, Tokmadzic et al 2002) by both maternal cells and the blastocyst itself, which in turn direct remodeling of endometrium at the site of implantation. Here we have shown the key role of TAB2 in mediating inflammatory signal-dependent derepression of E2-dependent repression of BMP7. Strikingly, BMP7 expression is locally induced in the tissue immediately adjacent to the implanting blastocyst, exactly at the putative site of IL-1 action in the implantation process (Paria et al 2001). Therefore local induction of estrogen-inhibited BMP7 levels by inflammatory signals may facilitate the changes in uterine tissue organization necessary for blastocyst invasion and implantation. The induction of parturition has recently been proposed to involve activated, IL-1β-producing macrophages recruited into the uterine wall in response to signals from the maturing fetus, an event which may also underlie pre-term labor invoked by uterine infection (Condon et al., 2004), it is tempting to speculate similar molecular mechanisms involving TAB2 and progesterone receptor-mediated events. Thus, it appears reasonable to suggest that the ability of inflammatory signals to reverse sex hormones dependent gene repression is an important biological strategy, related to reproductive and probably other critical aspects of mammalian homeostasis.

The present invention provides evidence for a macrophage/prostate cancer regulatory axis that causes derepression of SARM actions by selectively including TAB2 in the recruited N-CoR holocorepressor complex, setting the patterns of genome derepression events in response to pro-inflammatory signals. This finding has profound effects, including applications to resistance to SARMs/SERMs, and it implicates and exemplifies a powerful sensor-based strategy of integrating genome-wide responses to specific signaling pathways, apparently of particular physiological importance in reversing negative gene regulation events by the sex steroids. Defining this macrophage/cancer cell functional interaction as a key component in resistance also provides additional strategies to modify therapeutic approaches to specific cancers.

Even though antagonist/IL-1β and agonist both activate androgen receptor target genes, distinct molecular mechanisms act to regulate the transcriptional response, as variations of the association of various coactivator proteins were observed in response to different ligands. Most of the coactivator recruitment events were conserved in SARM and agonist-dependent activation. However, a striking difference was observed, in the SARM-dependent activation, as neither CBP/p300 nor CARM1 were recruited, or required for activation. While not being limited to any theories regarding the molecular bases of the invention, it is possible that these reflect on specific differences in the usage the three LXXLL (SEQ ID NO:27) helices (LXD) of the nuclear receptor interaction domains of the p160 factors and an allosteric mechanism (Alen et al., 1999; Bevan et al., 1999; Darimont et al., 1998; Nolte et al., 1998; Shiau et al., 1998; McInerney et al., 1998; He et al., 2002), because the LXD1 and LXD2 helices in SRC-1 are required for DHT-mediated activation, while the LXD3 and LXD2 helices in SRC-1 are required for the activation by SARMs. These findings are of particular interest because of the findings (Brzozowski et al., 1997; Heery et al., 1997; Torchia et al., 1997, McInerney et al., 1998; Darimont et al., 1998; Nolte et al., 1998; Shiau et al., 1998) that many nuclear receptor coactivators contain multiple LXXLL (SEQ ID NO:27) helical motifs. Despite this distribution in cofactor recruitment, DHT and SARMs prove to activate virtually identical transcriptional programs. Indeed, distinct LXXLL (SEQ ID NO:27) motifs are used in recruitment of RIP140 to various DNA-binding partners, which is again associated with allosteric effects of these resulting conformational differences in coactivator complexes (He et al., 2002; Christian et al., 2004). It is possible that TAB2 acts to “break” N- and C-terminal androgen receptor domain interaction, further enforcing the repressive state.

Distinctive sets of coactivators are not corecruited to androgen target genes tested, such as the HATs Tip60 and p300, the p160 factors p/CIP and SRC1, and the Brg1 component of the SWI/SNF complex and p300, revealing the reciprocal recruitment of specific group of coactivator complexes, quite reminiscent of finding of cohorts of corecruited complexes for the pS2 promoter in response to liganded ER (Metivier et al., 2003), suggesting that the alternative recruitment of these complexes may be a general property of many nuclear receptors. Because many of these complexes are also required for activation, there appears be a molecular strategy for temporal order of coactivator complex recruitment and dismissal, fulfilling predictions that ligand-specific interactions might result in distinct cofactor recruitment (McDonald et al., 1996).

It will be apparent to those skilled in the art that various modifications and variations can be made in the practice of the present invention without departing from the scope or spirit of the invention. Other embodiments of the invention will be apparent to those skilled in the art from consideration of the specification and practice of the invention. It is intended that the specification and examples be considered as exemplary only, with a true scope and spirit of the invention being indicated by the following claims.

### REFERENCES

The following references are cited in the application and provide general information on the field of the invention and provide assays and other details discussed in the application. The following references are incorporated herein for their disclosures, as they relate to the present invention.
Alen, P., Claessens, F., Verhoeven, G., Rombauts, W., and Peeters, B. (1999) The androgen receptor amino-terminal domain plays a key role in p160 coactivator-stimulated gene transcription. Mol. Cell. Biol. 19, 6085-6097.
Alland, L., Muhle, R., Hou, H. Jr., Potes, J., Chin, L., Schreiber-Agus, N., and DePinho, R. A. (1997) Role for N-CoR and histone deacetylase in Sin3-mediated transcriptional repression. Nature 387, 49-55.
Arkan, M. C., Hevener, A. L., Greten, F. R., Maeda, S., Li, Z. W., Long, J. M., Wynshaw-Boris, A., Poli, G., Olefsky, J., and Karin, M. (2005). IKK-b links inflammation to obesity-induced insulin resistance. Nat. Med. 11, 191-198.
Baek, S. H., Ohgi, K. A., Rose, D. W., Koo, E. H., Glass, C. K., and Rosenfeld, M. G. (2002) Exchange of N-CoR corepressor and Tip60 coactivator complexes links gene expression by NF-kB and b-amyloid precursor protein. Cell 110, 55-67.
Baek, S. H., and Rosenfeld, M. G. (2004) Nuclear receptor coregulators: their modification codes and regulatory mechanism by translocation. Biochem. Biophys. Res. Commun. 319, 707-714.
Balk, S. P. (2002) Androgen receptor as a target in androgen-independent prostate cancer. Urology. 60, 132-138.
Bannister, A. J., and Kouzarides, T. (1996) The CBP co-activator is a histone acetyltransferase. Nature 384, 641-643.
Berry, M., Metzger, D., and Chambon, P. (1990) Role of the two activating domains of the oestrogen receptor in the cell-type and promoter-context dependent agonistic activity of the anti-oestrogen 4-hydroxytamosifen. EMBO J. 9, 2811-2818.
Bevan, C. L., Hoare, S, Claessens, F., Heery, D. M., and Parker, M. G. (1999) The AF1 and AF2 domains of the androgen receptor interact with distinct regions of SRC1. Mol. Cell Biol. 19, 8383-8392.
Brownell, J. E., Zhou, J., Ranalli, T., Kobayashi, R., Edmondson, D. G., Roth, S. Y., and Allis, C. D. (1996) Tetrahymena histone acetyltransferase A: a homolog to yeast Gcn5p linking histone acetylation to gene activation. Cell 84, 843-851.
Brzozowski, A. M., Pike, A. C., Dauter, Z., Hubbard, R. E., Bonn, T., Engstrom, O., Ohman, L., Greene, G. L., Gustafsson, J. A., and Carlquist, M. (1997) Molecular basis of agonism and antagonism in the oestrogen receptor. Nature 389, 753-758.
Cai, D., Yuan, M., Frantz, D. F., Melendez, P. A., Hansen, L., Lee, J., and Shoelson, S. E. (2005). Local and systemic insulin resistance resulting from hepatic activation of IKK-b and NF-kB. Nat. Med. 11, 183-190.
Cavailles, V., Dauvois, S., L'Horset, F., Lopez, G., Hoare, S., Kushner, P. J., and Parker, M. G. (1995) Nuclear factor RIP140 modulates transcriptional activation by the estrogen receptor. EMBO J. 14, 3741-3751.
Chakravarti, D., LaMorte, V. J., Nelson, M. C., Nakajima, T., Schulman, I. G., Juguilon, H., Montminy, M., and Evans, R. M. (1996) Role of CBP/p300 in nuclear receptor signaling. Nature 383, 99-103.
Chen, C. D., Welsbie, D. S., Tran, C., Baek, S. H., Chen, R., Vessella, R., Rosenfeld, M. G., and Sawyers, C. L. (2004) Molecular determinants of resistance to antiandrogen therapy. Nat. Med. 10, 33-39.
Chen, D., Ma, H., Hong, H., Koh, S. S., Huang, S. M., Schurter, B. T., Aswad, D. W., and Stallcup, M. R. (1999) Regulation of transcription by a protein methyltransferase. Science 284, 2174-2177.
Chen, H., Lin, R. J., Schiltz, R. L., Chakravarti, D., Nash, A., Nagy, L., Privalsky, M. L., Nakatani, Y., and Evans, R. M. (1997) Nuclear receptor coactivator ACTR is a novel histone acetyltransferase and forms a multimeric activation complex with P/CAF and CBP/p300. Cell 90, 569-580.
Chen, J. D., and Evans, R. M. (1995) A transcriptional co-repressor that interacts with nuclear hormone receptors. Nature 377, 454-457.
Chin, W. W., Carr, F. E., Burnside, J., and Darling, D. S. (1993) Thyroid hormone regulation of thyrotropin gene expression. Recent Prog. Horm. Res. 48, 393-414.
Christian, M., Tullet, J. M., and Parker, M. G. (2004) Characterization of four autonomous repression domains in the corepressor receptor interacting protein 140. J. Biol. Chem. 279, 15645-15651.
Chung, T. D., Yu, J. J., Spiotto, M. T., Bartkowski, M., and Simons, J. W. (1999) Characterization of the role of IL-6 in the progression of prostate cancer. Prostate 38, 199-207.
Cleutjens, K. B., van Eekelen, C. C., van der Korput, H. A., Brinkmann, A. O., and Trapman, J. (1996) Two androgen response regions cooperate in steroid hormone regulated activity of the prostate-specific antigen promoter. J. Biol. Chem. 271, 6379-6388.
Cohen, P. E., Nishimura, K., Zhu, L., and Pollard, J. W. (1999) Macrophages: important accessory cells for reproductive function. J Leukoc Biol. 66, 765-762.
Condon, J. C., Jevasuria, P., Faust, J. M., and Mendelson, C. R. (2004) Surfactant Protein secreted by the maturing mouse fetal lung acts as a hormone that signals the initiation of parturition. Proc. Natl. Acad. Sci. U.S.A. 101, 4978-4983.
Coussens, L. M. and Werb, Z. (2002). Inflammation and cancer. Nature 420, 860-867.
Darimont, B. D., Wagner, R. L., Apriletti, J. W., Stallcup, M. R., Kushner, P J., Baxter, J. D., Fletterick, R. J., Yamamoto, K. R. (1998) Structure and specificity of nuclear receptor-coactivator interactions. Genes & Dev. 12, 3343-3356.
Debes, J. D., and Tindall, D. J. (2004). Mechanisms of androgen-refractory prostate cancer. N. Engl. J. Med. 351: 1488-1490.
De los Santos, M. J., Mercader, A., Frances, A., Portoles, E., Remohi, J., Pellicer, A., and Simon, C. (1996) Role of endometrial factors in regulating secretion of components of the immunoreactive human embryonic interleukin-1 system during embryonic development. Biol Reprod. 54, 563-574.
Ding, X. F., Anderson, C. M., Ma, H., Hong, H., Uht, R. M., Kushner, P. J., and Stallcup, M. R. (1998) Nuclear receptor-binding sites of coactivators glucocorticoid receptor interacting protein 1 (GRIP1) and steroid receptor coactivator 1 (SRC-1): multiple motifs with different binding specificities. Mol. Endocrinol. 12, 302-313.
Dostert, A. and Heinzel, T. (2004) Negative glucocorticoid receptor response elements and their role in glucocorticoid action. Curr. Pharm. Des. 10, 2807-2816.
Feldman, B. J., and Feldman, D. (2001) The development of androgen-independent prostate cancer. Nat. Rev. Cancer 1, 34-45.
Frasor, J., Danes, J. M., Komm, B., Chang, K. C., Lyttle, C. R., and Katzenellenbogen, B. S. (2003). Profiling of estrogen up- and down-regulated gene expression in human breast cancer cells: insights into gene networks and pathways underlying estrogenic control of proliferation and cell phenotype. Endocrinology 144: 4562-4574.
Gibson, S., Widmann, C., and Johnson, G. L. (1999) Differential involvement of MEK kinase 1 (MEKK1) in the induction of apoptosis in response to microtubule-targeted drugs versus DNA damaging agents. J. Biol. Chem. 274, 10916-10922.
Glass, C. K., and Rosenfeld, M. G. (2000) The coregulator exchange in transcriptional functions of nuclear receptors. Genes & Dev. 14, 121-141.
Gouon-Evans, V., Rothenberg, M. E., and Pollard, J. W. (2000) Postnatal mammary gland development requires macrophages and eosinophils. Development. 127, 2269-2282.
Grese, T. A., Sluka, J. P., Bryant, H. U., Cullinan, G. J., Glasebrook, A. L., Jones, C. D., Matsumoto, K., Palkowitz, A. D., Sato, M., Termine, J. D., Winter, M. A., Yang, N. N., Dodge, J. A. (1997) Molecular determinants of tissue selectivity in estrogen receptor modulators. Proc. Natl. Acad. Sci. USA. 94, 14105-14110.
Greten, F. R., Eckmann, L., Greten, T. F., Park, J. M., Li, Z. W., Egan, L. J., Kagnoff, M. F., and Karin, M. (2004). IKKb links inflammation and tumorigenesis in a mouse model of colitis-associated cancer. Cell 118: 285-296.
Guenther, M. G., Lane, W. S., Fischle, W., Verdin, E., Lazar, M. A., and Shiekhattar, R. (2000) A core SMRT corepressor complex containing HDAC3 and TBL1, a WD40-repeat protein linked to deafness. Genes & Dev. 4, 1048-1057.
Gustafsson, J. A. (1999) Estrogen receptor beta—a new dimension in estrogen mechanism of action. J. Endocrinol. 163, 379-383.
Hassig, C. A., and Schreiber, S. L. (1997) Nuclear histone acetylases and deacetylases and transcriptional regulation: HATs off to HDACs. Curr. Opin. Chem. Biol. 1, 300-308.
Hassig, C. A., Tong, J. K., Fleischer, T. C., Owa, T., Grable, P. G., Ayer, D. E., and Schreiber, S. L. (1998) A role for histone deacetylase activity in HDAC1-mediated transcriptional repression. Proc. Natl. Acad. Sci. USA. 95, 3519-3524.
He, B., Lee, L. W., Minges, J. T., and Wilson, E. M. (2002) Dependence of selective gene activation on the androgen receptor NH2- and COOH-terminal interaction. J. Biol. Chem. 277, 25631-25639.
Heery, D. M., Kallhoven, E., Hoare, S., and Parker, M. G (1997) A signature motif in transcriptional co-activators mediates binding to nuclear receptors. Nature 387, 733-736.
Heery D. M., Hoare S., Hussain, S., Parker, M. G., and Sheppard, H. (2001) Core LXXLL motif sequences in CREB-binding protein, SRC1, and RIP140 define affinity and selectivity for steroid and retinoid receptors. J. Biol. Chem. 276, 6695-6702.
Heinlein, C. A., and Chang, C. (2002) Androgen receptor (AR) coregulators: an overview. Endocr. Rev. 23, 175-200.
Heinzel, T., Lavinsky, R. M., Mullen, T. M., Soderstrom, M., Laherty, C. D., Torchia, J., Yang, W. M., Brard, G., Ngo, S. D., Davie, J. R., Seto, E., Eisenman, R. N., Rose, D. W., Glass, C. K., and Rosenfeld, M. G. (1997) A complex containing N-CoR, mSin3 and histone deacetylase mediates transcriptional repression. Nature 387, 43-48.
Hong, H., Kohli, K., Garabedian, M. J., and Stallcup, M. R. (1997) GRIP1, a transcriptional coactivator for the AF-2 transactivation domain of steroid, thyroid, retinoid, and vitamin D receptors. Mol. Cell Biol. 17, 2735-2744.
Holness, C. L., and Simmons, D. L. (1993). Molecular cloning of CD68, a human macrophage marker related to lysosomal glycoproteins. Blood 81(6): 1607-1613.
Horlein, A. J., Naar, A. M., Heinzel, T., Torchia, J., Gloss, B., Kurokawa, R., Ryan, A., Kamei, Y., Soderstrom, M., Glass, C. K., and Rosenfeld, M. G. (1995) Ligand-independent repression by the thyroid hormone receptor mediated by a nuclear receptor co-repressor. Nature 377, 397-404.
Hu, X., and Lazar, M. A. (1999) The CoRNR motif controls the recruitment of corepressors by nuclear hormone receptors. Nature 402, 93-96.
Humphrey G. W., Wang, Y., Russanova, V. R., Hirai, T., Qin, J., Nakatani, Y., and Howard, B. H. (2001) Stable histone deacetylase complexes distinguished by the presence of SANT domain proteins CoREST/kiaa0071 and Mta-L1. J. Biol. Chem. 276, 6817-6824.
Ikura, T., Ogryzko, V. V., Grigoriev, M., Groisman, R., Wang, J., Horikoshi, M., Scully, R., Qin, J., and Nakatani, Y. (2000) Involvement of the TIP60 histone acetylase complex in DNA repair and apoptosis. Cell 102, 463-473.
Jackson, T. A., Richer, J. K., Bain, D. L., Takimoto, G. S., Tung, L., and Horwitz, K. B. (1997) The partial agonist activity of antagonist-occupied steroid receptors is controlled by a novel hinge domain-binding coactivator L7/SPA and the corepressors N-CoR or SMRT. Mol. Endocrinol. 11, 693-705.
Jakacka, M., Ito, M., Weiss, J., Chien, P. Y., Gehm, B. D., and Jameson, J. L. (2001). Estrogen receptor binding to DNA is not required for its activity through the nonclassical AP1 pathway. J. Biol. Chem. 276: 13615-13621.
Jepsen, K., Hermanson, O., Onami, T. M., Gleiberman, A. S., Lunyak, V., McEvilly, R. J., Kurokawa, R., Kumar, V., Liu, F., Seto, E., Hedrick, S. M., Mandel, G., Glass, C. K., Rose, D. W., and Rosenfeld, M. G. (2000) Combinatorial roles of the nuclear receptor corepressor in transcription and development. Cell 102, 753-763.
Jepsen K, Rosenfeld M G. (2002). Biological roles and mechanistic actions of co-repressor complexes. J Cell Sci. 115:689-98.
Jonas, B. A. and Privalsky, M. L. (2004). SMRT and N-CoR corepressors are regulated by distinct kinase signaling pathways. J. Biol. Chem. 279: 54676-54686.
Kemppainen, J. A., Langley, E., Wong, C. I., Bobseine, K., Kelce, W. R., and Wilson, E. M. (1999) Distinguishing androgen receptor agonists and antagonists: distinct mechanisms of activation by medroxyprogesterone acetate and dihydrotestosterone. Mol. Endocrinol. 13, 440-454.
Kobayashi, Y., Kitamoto, T., Masuhiro, Y., Watanabe, M., Kase, T., Metzger, D., Yanagisawa, J., and Kato, S. (2000) p300 mediates functional synergism between AF-1 and AF-2 of estrogen receptor alpha and beta by interacting directly with the N-terminal A/B domains. J. Biol. Chem. 275, 15645-15651.
Kraus, W. L., McInerney, E. M., and Katzenellenbogen, B. S. (1995) Ligand-dependent, transcriptionally productive association of the amino- and carboxyl-terminal regions of a steroid hormone nuclear receptor. Proc. Natl. Acad. Sci. USA 92, 12314-12318.
Kusumegi, T., Tanaka, J., Kawano, M., Yonemoto, J., Tohyama, C., and Sone, H. (2004). BMP7/ActRIIB regulates estrogen-dependent apoptosis: new biomarkers for environmental estrogens. J. Biochem. Mol. Toxicol. 18: 1-11.
Langley, E., Zhou, Z. X., and Wilson, E. M. (1995) Evidence for an anti-parallel orientation of the ligand-activated human androgen receptor dimer. J Biol. Chem. 270, 29983-29990.
Lavinsky, R. M., Jepsen, K., Heinzel, T., Torchia, J., Mullen, T. M., Schiff, R., Del-Rio, A. L., Ricote, M., Ngo, S., Gemsch, J., Hilsenbeck, S. G., Osborne, C. K., Glass, C. K., Rosenfeld, M. G., and Rose, D. W. (1998) Diverse signaling pathways modulate nuclear receptor recruitment of N-CoR and SMRT complexes. Proc. Natl. Acad. Sci. USA 95, 2920-2925.
Le Douarin, B., Zechel, C., Garnier, J. M., Lutz, Y., Tora, L., Pierrat, P., Heery, D., Gronemeyer, H., Chambon, P., and Losson, R. (1995) The N-terminal part of TIF1, a putative mediator of the ligand-dependent activation function (AF-2) of nuclear receptors, is fused to B-raf in the oncogenic protein T18. EMBO J. 14, 2020-2033.
Leek, R. D., Lewis, C. E., Whitehouse, R., Greenall, M., Clarke, J., and Harris, A. L. (1996). Association of macrophage infiltration with angiogenesis and prognosis in invasive breast carcinoma. Cancer Res. 56: 4625-4629.
Leid, M., Kastner, P., and Chambon, P. (1992) Multiplicity generates diversity in the retinoic acid signalling pathways. Trends Biochem. Sci. 17, 427-433.
Li, A. C. and Glass, C. K. (2002) The macrophage foam cell as a target for therapeutic intervention. Nat. Med. 8, 1235-1242.
Li, J., Wang, J., Wang, J., Nawaz, Z., Liu, J. M., Qin, J., and Wong, J. (2000) Both corepressor proteins SMRT and N-CoR exist in large protein complexes containing HDAC3. EMBO J. 19, 4342-4350.
Lin, C. Y., Strom, A., Vega, V. B., Kong, S. L., Yeo, A. L., Thomsen, J. S., Chan, W. C., Doray, B., Bangarusamy, D. K., Ramasamy, A., Vergara, L. A., Tang, S., Chong, A., Bajic, V. B., Miller, L. D., Gustafsson, J. A., and Liu, E. T. (2004). Discovery of estrogen receptor alpha target genes and response elements in breast tumor cells. Genome Biol. 5: R66.
Lu, Z., Xu, S., Joazeiro, C., Cobb, M. H., Hunter, T. (2002). The PHD domain of MEKK1 acts as an E3 ubiquitin ligase and mediates ubiquitination and degradation of ERK1/2. Mol Cell. 9:945-56.
Martinez-Pomares, L., Platt, N., McKnight, A. J., da Silva, R. P., and Gordon, S. (1996). Macrophage membrane molecules: markers of tissue differentiation and heterogeneity. Immunobiology 195: 407-416.
McDonnell, D. P., and Norris, J. D. (2002) Connections and regulation of the human estrogen receptor. Science 296, 1642-1644.
McInerney, E. M., Rose, D. W., Flynn, S. E., Westin, S., Mullen, T. M., Krones, A., Inostroza, J., Torchia, J., Nolte, R. T., Assa-Munt, N., Milburn, M. V., Glass, C. K., and Rosenfeld, M. G. (1998) Determinants of coactivator LXXLL motif specificity in nuclear receptor transcriptional activation. Genes & Dev 12, 3357-3368.
McKenna, N. J., and O'Malley, B. W. (2002) Combinatorial control of gene expression by nuclear receptors and coregulators. Cell 108, 465-474.
Metivier, R., Penot, G., Flouriot, G., and Pakdel, F. (2001) Synergism between ERalpha trans activation function 1 (AF-1) and AF-2 mediated by steroid receptor coactivator protein-1: requirement for the AF-1 alpha-helical core and for a direct interaction between the N- and C-terminal domains. Mol. Endocrinol. 15, 1953-1970.
Metivier, R., Penot, G., Hubner, M. R., Reid, G., Brand, H., Kos, M., and Gannon, F. (2003) Estrogen receptor-a directs ordered, cyclical, and combinatorial recruitment of cofactors on a natural target promoter. Cell 115, 751-763.
Mizzen, C. A., Yang, X. J., Kokubo, T., Brownell, J. E., Bannister, A. J., Owen-Hughes, T., Workman, J., Wang, L., Berger, S. L., Kouzarides, T., Nakatani, Y., and Allis, C. D. (1996) The TAF(II) 250 subunit of TFIID has histone acetyltransferase activity. Cell 87, 1261-1270.
Monroe, D. G., Jin, D. F., and Sanders, M. M. (2000). Estrogen opposes the apoptotic effects of bone morphogenetic protein 7 on tissue remodeling. Mol. Cell. Biol. 20: 4626-4634.
Moras, D., and Gronemeyer, H. (1998) The nuclear receptor ligand-binding domain: structure and function. Curr. Opin. Cell. Biol. 10, 384-391.
Nagy, L., Kao, H. Y., Chakravarti, D., Lin, R. J., Hassig, C. A., Ayer, D. E., Schreiber, S. L., and Evans, R. M. (1997) Nuclear receptor repression mediated by a complex containing SMRT, mSin3A, and histone deacetylase. Cell 89, 373-380.
Nagy, L., Kao, H. Y., Love, J. D., Li, C., Banayo, E., Gooch, J. T., Krishna, V., Chatterjee, K., Evans, R. M., and Schwabe, J. W. R. (1999) Mechanism of corepressor binding and release from nuclear hormone receptors. Genes & Dev. 13, 3209-3216.
Nolte, R. T., Wisely, G. B., Westin, S., Cobb, J. E., Lambert, M. H., Kurokawa, R., Rosenfeld, M. G., Willson, T. M., Glass, C. K., and Milburn, M. V. (1998) Ligand binding and co-activator assembly of the peroxisome proliferator-activated receptor-g. Nature 395, 137-143.
Ogryzko, V. V., Schiltz, R. L., Russanova, V., Howard, B. H., and Nakatani, Y. (1996) The transcriptional coactivators p300 and CBP are histone acetyltransferases. Cell. 87, 953-959. Onate, S. A., Boonyaratanakomkit, V., Spencer, T. E., Tsai, S. Y., Tsai, M. J., Edwards, D. P., and O'Malley, B. W. (1998) The steroid receptor coactivator-1 contains multiple receptor interacting and activation domains that cooperatively enhance the activation function 1 (AF1) and AF2 domains of steroid receptors. J. Biol. Chem. 273, 12101-12108.
Ostojic, S., Dubanchet, S., Chaouat, G., Abdelkarim, M., Truyens, C., and Capron, F. (2003) Demonstration of the presence of IL-16, IL-17, and IL-18 at the murine fetomaternal interface during murine pregnancy. Am J Reprod Immunol. 49, 101-112.
Paria, B. C., Ma, W., Tan, J., Raja, S., Das, S. K., Dey, S. K., and Hogan, B. L. (2001). Cellular and molecular responses of the uterus to embryo implantation can be elicited by locally applied growth factors. Proc. Natl. Acad. Sci. U.S.A. 98: 1047-1052.
Perissi, V., Staszewski, L. M., McInerney, E. M., Kurokawa, R., Krones, A., Rose, D. W., Lambert, M. H., Milburn, M. V., Glass, C. K., and Rosenfeld, M. G. (1999) Molecular determinants of nuclear receptor-corepressor interaction. Genes & Dev. 13, 3198-3208.
Perissi, V., Aggarwal, A., Glass, C. K., Rose, D. W., and Rosenfeld, M. G. (2004). A corepressor/coactivator exchange complex required for transcriptional activation by nuclear receptors and other regulated transcription factors. Cell 116: 511-526.
Perlmann, T., and Evans, R. M. (1997) Nuclear receptors in Sicily: all in the famiglia. Cell. 90, 391-397.
Quigley, C. A., De Bellis, A., Marschke, K. B., el-Awady, M. K., Wilson, E. M., and French, F. S. (1995) Androgen receptor defects: historical, clinical, and molecular perspectives. Endocr. Rev. 16, 271-321.
Reid, J., Kelly, S. M., Watt, K., Price, N. C., McEwan, I. J. (2002). Conformational analysis of the androgen receptor amino-terminal domain involved in transactivation. Influence of structure-stabilizing solutes and protein-protein interactions. J. Biol. Chem. 277: 20079-20086.
Renaud, J. P., Rochel, N., Ruff, M., Vivat, V., Chambon, P., Gronemeyer, H., and Moras, D. (1995) Crystal structure of the RAR-g ligand-binding domain bound to all-trans retinoic acid. Nature 378, 681-689.
Sande, S., and Privalsky, M. L. (1996) Identification of TRACs (T3 receptor-associating cofactors), a family of cofactors that associate with, and modulate the activity of, nuclear hormone receptors. Mol. Endocrinol. 10, 813-825.
Sanjo, H., Takeda, K., Tsujimura, T., Ninomiya-Tsuji, J., Matsumoto, K., and Akira S. (2003) TAB2 is essential for prevention of apoptosis in fetal liver but not for interleukin-1 signaling. Mol. Cell Biol. 23, 1231-1238.
Shang, Y., Hu, X., DiRenzo, J., Lazar, M. A., and Brown, M. (2000) Cofactor dynamics and sufficiency in estrogen receptor-regulated transcription. Cell 103, 843-852.
Shang, Y., Myers, M., and Brown, M. (2002) Formation of the androgen receptor transcription complex. Mol. Cell 9, 601-610.
Shang, Y., and Brown, M. (2002) Molecular determinants for the tissue specificity of SERMs. Science 295, 2465-2468.
Shiau, A. K., Barstad, D., Loria, P. M., Cheng, L., Kushner, P. J., Agard, D. A., and Greene, G. L. (1998) The structural basis of estrogen receptor/coactivator recognition and the antagonism of this interaction by tamoxifen. Cell 95, 927-937.
Shimura, S., Yang, G., Ebara, S., Wheeler, T. M., Frolov, A., and Thompson, T. C. (2000). Reduced infiltration of tumor-associated macrophages in human prostate cancer: association with cancer progression. Cancer Res. 60: 5857-5861.
Smith, C. L., Nawaz, Z., and O'Malley, B. W. (1997) Coactivator and corepressor regulation of the agonist/antagonist activity of the mixed antiestrogen, 4-hydroxytamoxifen. Mol. Endocrinol. 11, 657-666.
Smith, C. L. and O'Malley, B. W. (2004) Coregulator function: a key to understanding tissue specificity of selective receptor modulators. Endocr. Rev. 25, 45-71.
Spencer, T. E., Jenster, G., Burcin, M. M., Allis, C. D., Zhou, J., Mizzen, C. A., McKenna, N. J., Onate, S. A., Tsai, S. Y., Tsia, M. J., and O'Malley, B. W. (1997) Steroid receptor coactivator-1 is a histone acetyltransferase. Nature 389, 194-198.
Takaesu, G., Kishida, S., Hiyama, A., Yamaguchi, K., Shibuya, H., Irie, K., Ninomiya-Tsuji, J., and Matsumoto, K. (2000) TAB2, a novel adaptor protein, mediates activation of TAK1 MAPKKK by linking TAK1 to TRAF6 in the IL-1 signal transduction pathway. Mol. Cell 5, 649-658.
Tetel, M. J., Giangrande, P. H., Leonhardt, S. A., McDonnell, D. P., and Edwards, D. P. (1999) Hormone-dependent interaction between the amino- and carboxyl-terminal domains of progesterone receptor in vitro and in vivo. Mol. Endocrinol. 13, 910-924.
Tolmadzic, V. S., Tsuji, Y., Bogovic, T., Laskarin, G., Cupurdija, K., Strbo, N., Koyama, K., Okamura, H., Podack, E. R., and Rukavina, D. (2002) IL-18 is present at the maternal-fetal interface and enhances cytotoxic activity of decidual lymphocytes. Am J Reprod Immunol. 48, 191-200.
Tong, J. K., Hassig, C. A., Schnitzler, G. R., Kingston, R. E., and Schreiber, S. L. (1998) Chromatin deacetylation by an ATP-dependent nucleosome remodelling complex. Nature 395, 917-921.
Torchia, J., Rose, D. W., Inostroza, J., Kamei, Y., Westin, S., Glass, C. K., and Rosenfeld, M. G. (1997) The transcriptional coactivator p/CIP binds CBP and mediates nuclear-receptor function. Nature 387, 677-684.
Tremblay, A., Tremblay, G. B., Labrie, F., and Giguere, V. (1999) Ligand-independent recruitment of SRC-1 to estrogen receptor beta through phosphorylation of activation function AF-1. Mol. Cell. 3, 513-519.
Underhill, C., Qutob, M. S., Yee, S. P., and Torchia, J. (2000) A novel nuclear receptor corepressor complex, N-CoR, contains components of the mammalian SWI/SNF complex and the corepressor KAP-1. J. Biol. Chem. 275, 40463-40470.
Voegel, J. J., Heine, M. J., Tini, M., Vivat, V., Chambon, P., and Gronemeyer, H. (1998) The coactivator TIF2 contains three nuclear receptor-binding motifs and mediates transactivation through CBP binding-dependent and -independent pathways. EMBO J. 17, 507-519.
Wagner, R. L., Apriletti, J. W., McGrath, M. E., West, B. L., Baxter, J. D., and Fletterick, R. J. (1995) A structural role for hormone in the thyroid hormone receptor. Nature 378, 690-697.
Webb, P., Nguyen, P., and Kushner, P. J. (2003) Differential SERM effects on corepressor binding dictate ERalpha activity in vivo. J. Biol. Chem. 278, 6912-6920.
Weisberg, S. P., McCann, D., Desai, M., Rosenbaum, M., Leibel, R. L., and Ferrante, A. W. Jr. (2003). Obesity is associated with macrophage accumulation in adipose tissue. J. Clin. Invest. 112: 1796-1808.
Wen, Y.-D., Perissi, V., Staszewski, L. M., Yang, W.-M., Krones, A., Glass, C. K., Rosenfeld, M. G., and Seto, E. (2000) The histone deacetylase-3 complex contains nuclear receptor corepressors. Proc. Natl. Acad. Sci. USA 97, 7202-7207.
Wu, Y. L., Yang, X., Ren, Z., McDonnell, D. P, Norris, J. D., Willson, T. M., and Greene, G. L. (2005). Structural basis for an unexpected mode of SERM-mediated ER antagonism. Mol. Cell. 18: 413-424.
Xu, H., Barnes, G. T., Yang, Q., Tan, G., Yang, D., Chou, C. J., Sole, J., Nichols, A., Ross, J. S., Tartaglia, L. A., and Chen, H. (2003). Chronic inflammation in fat plays a crucial role in the development of obesity-related insulin resistance. J. Clin. Invest. 112:1821-1830.
Yang, X. J., Ogryzko, V. V., Nishikawa, J., Howard, B. H., and Nakatani, Y (1996) A p300/CBP-associated factor that competes with the adenoviral oncoprotein E1A. Nature 382, 319-324.
Yoon H. G., Chan D. W., Huang Z. Q., Li J., Fondell J. D., Qin J., Wong J. (2003). Purification and functional characterization of the human N-CoR complex: the roles of HDAC3, TBL1 and TBLR1. EMBO J. 22(6):1336-1346
Zhang, J., Kalkum, M., Chait, B. T., and Roeder, R. G. (2002) The N-CoR-HDAC3 nuclear receptor corepressor complex inhibits the JNK pathway through the integral subunit GPS2. Mol. Cell 9, 611-623.
Zhang, Y., Ng, H. H., Erdjument-Bromage, H., Tempst, P., Bird, A., and Reinberg, D. (1999) Analysis of the NuRD subunits reveals a histone deacetylase core complex and a connection with DNA methylation. Genes Dev. 13, 1924-1935.
Zhu, P., Martin, E., Mengwasser, J., Schlag, P., Janssen, K. P., and Gottlicher, M. (2004). Induction of HDAC2 expression upon loss of APC in colorectal tumorigenesis. Cancer Cell 5: 455-463.

1. A method of detecting a disease or disorder, said method comprising:
obtaining a sample comprising biological material; and
detecting in the sample the presence of at least one substance involved in a macrophage-induced signaling pathway leading to gene transcription,
wherein the substance is not produced by the macrophage.

2. The method of claim 1, wherein the disease or disorder is hormone-related.

3. The method of claim 1, wherein the disease or disorder is a cancer.

4. The method of claim 1, wherein the disease or disorder is prostate or breast cancer, diabetes, or atherosclerosis.

5. The method of claim 1, wherein the substance is at least one VCAM1 molecule.

6. The method of claim 1, wherein the substance is at least one MEKK1 molecule.

7. The method of claim 1, wherein the substance is at least one TAB2 molecule.

8. The method of claim 7, wherein the TAB2 molecule is phosphorylated.

9. The method of claim 1, wherein the substance is at least one N-CoR complex or a component of an N-CoR complex.

10. The method of claim 1, further comprising detecting the presence of at least one macrophage in the sample.

11. A method of treating a disease or disorder, said method comprising administering to a subject suffering from said disease or disorder at least one substance that specifically affects expression or activity of a protein in a macrophage-induced signaling pathway in a cell of the disease or disorder.

12. The method of claim 11, wherein the signaling pathway leads to regulation of a hormone receptor-regulated gene.

13. The method of claim 11, wherein the disease or disorder is a cancer.

14. The method of claim 11, wherein the disease or disorder is prostate cancer, breast cancer, diabetes, or atherosclerosis.

15. The method of claim 11, wherein the subject is human.

16. The method of claim 11, wherein the substance is an inhibitor of MEKK1 expression or an inhibitor of MEKK1 binding activity to a complex comprising an N-CoR protein.

17. The method of claim 11, wherein the substance is an inhibitor of VCAM1 expression or an inhibitor of VCAM1-mediated binding of macrophages to other cells.

18. The method of claim 11, wherein the substance is an inhibitor of TAB2 expression, an inhibitor of phosphorylation of TAB2, or an inhibitor of TAB2 binding to or interaction with a complex comprising N-CoR.

19. The method of claim 11, wherein the substance is a peptide, polypeptide, or protein comprising an amino acid sequence having at least one L/HX₇LL motif or a peptidomimetic of this motif.

20. The method of claim 11, wherein the method is a method of inhibiting or reversing insensitivity to one or more agents that treat the disease or disorder.

21. A method of treating a female to affect fertility, said method comprising:
administering a substance that affects the interaction of at least one macrophages with at least one cell of a blastocyst or with a uterine cell at the site of attachment of a blastocyst to a uterine wall.

22. The method of claim 21, wherein the substance reduces or eliminates contact between a macrophage and a VCAM1 molecule on the blastocyst.

23. The method of claim 21, wherein the substance is an anti-antigen of TAB2.

24. A method of screening for one or more substances that are effective in treating a disease or disorder, said method comprising;
contacting at least one gene or gene expression product involved in a macrophage-induced pathway with at least one substance; and
determining if the substance affects the expression or activity of the gene or gene expression product, or if the substance affects the interaction of the gene expression product with another molecule in the pathway,
wherein the macrophage-induced pathway is a pathway in a cell other than the macrophage.

25. The method of claim 24, wherein the disease or disorder is hormone regulated.

26. The method of claim 24, wherein the disease or disorder is cancer.

27. The method of claim 24, wherein the disease or disorder is prostate or breast cancer, diabetes, or atherosclerosis.

28. The method of claim 24, wherein the gene or gene expression product is MEKK1.

29. The method of claim 24, wherein the gene or gene expression product is VCAM1.

30. The method of claim 24, wherein the method identifies substances that affect the interaction of VCAM1 and macrophages.

31. The method of claim 24, wherein the gene or gene expression product is TAB2.

32. The method of claim 24, wherein the method identifies substances that affect the interaction or binding of TAB2 and the N-CoR complex.

33. The method of claim 24, wherein the gene or gene expression product is part of the N-CoR complex.

34. The method of claim 30, wherein the substance comprises at least one L/HX₇LL motif or a peptidomimetic of this motif.

35. A method of screening for substances that treat a disease or disorder, said method comprising:
contacting at least one N-CoR−/− cell to a substance, and
determining the levels of expression of at least one genes, or the activity of at least one gene product, involved in a macrophage-induced signaling pathway.

36. The method of claim 35, wherein the N-CoR −/− cell is from or makes up a knock-out animal.
